SEQUENCE LISTING (14 sequences)

SEQ ID NO: 1 - protein, 237 aa, synthetic construct
  REGION 1..237: note = hypothetical DegU protein screening sequence
MENKASRKIKVLLADDHQLFREGLKRILNMEDDLEVIGECGDGIQVLEFCNQNKPDVVLMDINMPIENGVEATERLREIFPDIKVIILSIHDDESYVFETLRKGASGYLLKDMEAESLINAIRSVVEGHAYIHPKVTGKLINQLRRMTYLDETGAASGAAKEAGVKFVAGDNNPLTRREAEVLRLMAEGKSNKMIGEFLFISEKTVKNHVSSILQKMEVDDRTQAVINSIKNGWVTL

SEQ ID NO: 2 - protein, 384 aa, synthetic construct
  REGION 1..384: note = hypothetical DegS protein screening sequence
MDFQADAIDRVIKNAIEVMEDSKYQIFEICESARQELESLNQELQQVLKETAETIDKVDQLELDYRRSRIRLTEVSRDFVRYKEEDIKQAYEKATQLQLDLMIYREKEMYLKARRDDLQKRVRNVEKSVERAETIGSQMNVVLEYLSGDLNQVTRILESAKNRQLIGLKIILAQEEERKRIAREIHDGPAQSLANLVLRTEIVERMLAKQEFEMVQDELVDLKGQVRSGLEEVRKIIFNLRPMALDDLGLVPTLRKYVQDFEEKTKIRTTFETRGKEHRLPSAMEAAIFRLVQEALSNAAKHAYATYVSVEITYQAQMVKIVVKDNGVGFDVELLEQKSKSHFGLIGMRERVELLEGRMEIESAEGQGTKIVIHIPINVEKRKE

SEQ ID NO: 3 - protein, 260 aa, synthetic construct
  REGION 1..260: note = hypothetical Spo0A protein screening sequence
MQKIKVCLADDNRELVNLLEEYISSQEDMEVIGVAYNGQECLELLEEKVPDVLILDIIMPHLDGLAVLERLREMNLSPQPKVIMLTAFGQEDVTKKAVELGASYYILKPFDMENLANRIRQVSGSSSVSSSSSSSNVVPEGKPKNLDASITSIIHEIGVPAHIKGYMYLREAITMVYNDIELLGSITKVLYPDIAKKFNTTASRVERAIRHAIEVAWSRGNIDSISSLFGYTVSMSKAKPTNSEFIAMVADKLRIEHKVS

SEQ ID NO: 4 - DNA, 20 bp, synthetic construct
  source 1..20: mol_type = other DNA
  misc_feature 1..20: note = artificial sequence for creating a degU Q218* mutation by changing nucleotide position 652 C to T
cagcagtattttgcaaaaaa

SEQ ID NO: 5 - DNA, 20 bp, synthetic construct
  source 1..20: mol_type = other DNA
  misc_feature 1..20: note = artificial sequence for creating a degU M220N (i.e. DegU D223*+M220N+E221G+V222G) mutation by inserting an A a nucleotide pos. 658
cagcagtattttgcaaaaaa

SEQ ID NO: 6 - DNA, 20 bp, synthetic construct
  source 1..20: mol_type = other DNA
  misc_feature 1..20: note = artificial sequence for creating a degS L99F mutation by changing nucleotide pos. 295 C to T
tgtgatgattttccgcgaga

SEQ ID NO: 7 - DNA, 20 bp, synthetic construct
  source 1..20: mol_type = other DNA
  misc_feature 1..20: note = artificial sequence for creating a spo0A A257V mutation by changing nucleotide pos. 770 C to T
taagctgagaattgagaaca

SEQ ID NO: 8 - DNA, 804 bp, Paenibacillus polymyxa
  source 1..804: mol_type = unassigned DNA
  misc_feature 1..804: note = DSM365 spo0A wild type
ttgcaaaaaattgaggtattgttggctgatgacaaccgggaatttacgaatttgcttgctgaatatatttccgatcaggaggatatggaagttacaggaatcgcctataacggcgaagaagtgctccaacgcatcgcagaatcccgcaacatacctgatgtacttattttagatattattatgcctcatctggatggtctcggtgtattggagcgtttaagagaaatgaacctgactccacagccgaaaatcattatgctgactgcattcggtcaagaaaatattacacagagggccgtacagctcggggcatcttattatattttgaagccgtttgacatggaagtgctggctaaccgtgttcgccaattagtggggccgcagttagttagcagcagtccggtgaccatttcatccatgcggtccaacgttgtacctatgggtaagacgaaaaacctggatgccagtatcacggccatcatacatgaaattggtgtaccagctcacatcaagggctatcaatatttacgcgaagccattacgatggtgtacaataatatcgaaattttgggagccatcaccaaaacattatatccggcaatcgccgaaaagttcaaaacgacggcatctcgcgtagaacgcgccattcgtcatgctatcgaggtagcatggacccgtggaaatatcgacagcatttcacacctgttcgggtacaccattaatatcagtaagtccaaaccaaccaattcagagttcatagcgatggtagcagataagctgagaattgagaacaaggtgtcctga

SEQ ID NO: 9 - DNA, 804 bp, synthetic construct
  source 1..804: mol_type = other DNA
  misc_feature 1..804: note = DSM365 spo0A A257V, pos. 770 C to T
ttgcaaaaaattgaggtattgttggctgatgacaaccgggaatttacgaatttgcttgctgaatatatttccgatcaggaggatatggaagttacaggaatcgcctataacggcgaagaagtgctccaacgcatcgcagaatcccgcaacatacctgatgtacttattttagatattattatgcctcatctggatggtctcggtgtattggagcgtttaagagaaatgaacctgactccacagccgaaaatcattatgctgactgcattcggtcaagaaaatattacacagagggccgtacagctcggggcatcttattatattttgaagccgtttgacatggaagtgctggctaaccgtgttcgccaattagtggggccgcagttagttagcagcagtccggtgaccatttcatccatgcggtccaacgttgtacctatgggtaagacgaaaaacctggatgccagtatcacggccatcatacatgaaattggtgtaccagctcacatcaagggctatcaatatttacgcgaagccattacgatggtgtacaataatatcgaaattttgggagccatcaccaaaacattatatccggcaatcgccgaaaagttcaaaacgacggcatctcgcgtagaacgcgccattcgtcatgctatcgaggtagcatggacccgtggaaatatcgacagcatttcacacctgttcgggtacaccattaatatcagtaagtccaaaccaaccaattcagagttcatagcgatggtagtagataagctgagaattgagaacaaggtgtcctga

SEQ ID NO: 10 - DNA, 723 bp, Paenibacillus polymyxa
  source 1..723: mol_type = unassigned DNA
  misc_feature 1..723: note = DSM365 degU wild type
atggaaaatcaagaaattagcaatgcacccattaaagttctcttagcggacgatcatcagttgttccgcgaaggacttaaacgtattttgaatatggaggacgacattgaggtcatcggagaatgtggcgatggtattcaagtgttggaattctgtaacgtagagaaaccggatattgttctgatggacattaatatgccaatcgaaaatggtgtagaggcaactgaaaaactgcgtgagatgttcccggatgtcaaagttatcattttatccattcatgatgatgaaagctatgtatttgagacgttgcgcaagggagccaatggttatctgttaaaggatatggaggccgagtccctcattaatgctattcgttcggttcatgaaggctatgcgtttatccatccgaaggtaacaggtaagctcattcagcaactccgccggatgacctacttgaatgaaacaggggctatggctgaaggtcatacgaaggaagccggcgtgaagtttgtcgcaggtgaaaataatccattgacccgtcgtgaagctgaagtattgcgtctgatggctgaaggtaagagcaacaagatgatcggtgaatatttattcattagtgagaaaacggtcaaaaaccatgtcagcagtattttgcaaaaaatggaggttgatgaccgtacacaagcagttattaactcaatcaaatatggatgggttacgctgtaa

SEQ ID NO: 11 - DNA, 724 bp, synthetic construct
  source 1..724: mol_type = other DNA
  misc_feature 1..724: note = DSM365 degU D223*+M220N+E221G+V222G, pos. 658 A to AA
atggaaaatcaagaaattagcaatgcacccattaaagttctcttagcggacgatcatcagttgttccgcgaaggacttaaacgtattttgaatatggaggacgacattgaggtcatcggagaatgtggcgatggtattcaagtgttggaattctgtaacgtagagaaaccggatattgttctgatggacattaatatgccaatcgaaaatggtgtagaggcaactgaaaaactgcgtgagatgttcccggatgtcaaagttatcattttatccattcatgatgatgaaagctatgtatttgagacgttgcgcaagggagccaatggttatctgttaaaggatatggaggccgagtccctcattaatgctattcgttcggttcatgaaggctatgcgtttatccatccgaaggtaacaggtaagctcattcagcaactccgccggatgacctacttgaatgaaacaggggctatggctgaaggtcatacgaaggaagccggcgtgaagtttgtcgcaggtgaaaataatccattgacccgtcgtgaagctgaagtattgcgtctgatggctgaaggtaagagcaacaagatgatcggtgaatatttattcattagtgagaaaacggtcaaaaaccatgtcagcagtattttgcaaaaaaatggaggttgatgaccgtacacaagcagttattaactcaatcaaatatggatgggttacgctgtaa

SEQ ID NO: 12 - DNA, 723 bp, synthetic construct
  source 1..723: mol_type = other DNA
  misc_feature 1..723: note = DSM365 degU Q218*, pos. 652 C to T
atggaaaatcaagaaattagcaatgcacccattaaagttctcttagcggacgatcatcagttgttccgcgaaggacttaaacgtattttgaatatggaggacgacattgaggtcatcggagaatgtggcgatggtattcaagtgttggaattctgtaacgtagagaaaccggatattgttctgatggacattaatatgccaatcgaaaatggtgtagaggcaactgaaaaactgcgtgagatgttcccggatgtcaaagttatcattttatccattcatgatgatgaaagctatgtatttgagacgttgcgcaagggagccaatggttatctgttaaaggatatggaggccgagtccctcattaatgctattcgttcggttcatgaaggctatgcgtttatccatccgaaggtaacaggtaagctcattcagcaactccgccggatgacctacttgaatgaaacaggggctatggctgaaggtcatacgaaggaagccggcgtgaagtttgtcgcaggtgaaaataatccattgacccgtcgtgaagctgaagtattgcgtctgatggctgaaggtaagagcaacaagatgatcggtgaatatttattcattagtgagaaaacggtcaaaaaccatgtcagcagtattttgtaaaaaatggaggttgatgaccgtacacaagcagttattaactcaatcaaatatggatgggttacgctgtaa

SEQ ID NO: 13 - DNA, 1161 bp, synthetic construct
  source 1..1161: mol_type = other DNA
  misc_feature 1..1161: note = DSM365 degS wild type
gtggactttcaagccgatatcatagaccgagtcattaaaaatgccatccaggtgatggagaacagcaaatatcagatgttcgaaatattggacacggcccggaccgagctggtcacgttaaatcaggagctccagagcgtcttgaaggaaacagcagaaacgatcgaaaaggtggaccagctggaaatgaactatcggcggtcccgcattcggttgactgaggtcagccgtgacttcgttcgctattcggaagaggatatcaagcaggcttatgagaaggcaacacagcttcaactcgatgtgatgattttccgcgagaaggaaatgtatctcaaagccagaagagatgatcttcaaaagcgggccaaaagtgttgaggcttctgtcgagcgcgccgaaaccatcggttcgcagatgggagtcgtgctggaatatttgtcgggcgaattgggacaagtgacgcggatcatcgaatcggctaaaaaccggcagtttattggtctgaaaattattttggctcaggaagaggagcgcaaacgcatatcccgtgaaattcacgatggacctgcgcagctccttgcgcatctagtgcttaggacggaaattgtggaaagaatgatcgccaagcaggaatttaagatggttcaggacgaaatagtagacttgaagaaacaggttcgctccagtcttgaggaaatgcgaaaggtcattttcaatttgcgtcctatggccctggatgacctgggacttattccgacgctccggaaatatgtgcaggattttgaagaaaaaacgaagattagatcgctttttgaaacaaggggcaaggaacaccgtctctcttccgcgatggaagcagccatttaccgtctgatccaggaagctttgaccaatgctgccaagcatgcctatcctacctatgtgcttgttgagattacctatcaggcgcagcttgtaaaaatcgtggtgcaggataatggtttgggctttaagccagagctttttcagcaaaaaagcaaagatcatgggcattttggtctgattggtatgcgggaaagggttgaactgctcgaggggagaatggagatcgaatcagctgagaatcaaggcaccaagatagtgattcatatcccaaccaacgtggaaaagggaaaggagtaa

SEQ ID NO: 14 - DNA, 1161 bp, synthetic construct
  source 1..1161: mol_type = other DNA
  misc_feature 1..1161: note = DSM365 degS L99F, pos. 295 C to T
gtggactttcaagccgatatcatagaccgagtcattaaaaatgccatccaggtgatggagaacagcaaatatcagatgttcgaaatattggacacggcccggaccgagctggtcacgttaaatcaggagctccagagcgtcttgaaggaaacagcagaaacgatcgaaaaggtggaccagctggaaatgaactatcggcggtcccgcattcggttgactgaggtcagccgtgacttcgttcgctattcggaagaggatatcaagcaggcttatgagaaggcaacacagcttcaattcgatgtgatgattttccgcgagaaggaaatgtatctcaaagccagaagagatgatcttcaaaagcgggccaaaagtgttgaggcttctgtcgagcgcgccgaaaccatcggttcgcagatgggagtcgtgctggaatatttgtcgggcgaattgggacaagtgacgcggatcatcgaatcggctaaaaaccggcagtttattggtctgaaaattattttggctcaggaagaggagcgcaaacgcatatcccgtgaaattcacgatggacctgcgcagctccttgcgcatctagtgcttaggacggaaattgtggaaagaatgatcgccaagcaggaatttaagatggttcaggacgaaatagtagacttgaagaaacaggttcgctccagtcttgaggaaatgcgaaaggtcattttcaatttgcgtcctatggccctggatgacctgggacttattccgacgctccggaaatatgtgcaggattttgaagaaaaaacgaagattagatcgctttttgaaacaaggggcaaggaacaccgtctctcttccgcgatggaagcagccatttaccgtctgatccaggaagctttgaccaatgctgccaagcatgcctatcctacctatgtgcttgttgagattacctatcaggcgcagcttgtaaaaatcgtggtgcaggataatggtttgggctttaagccagagctttttcagcaaaaaagcaaagatcatgggcattttggtctgattggtatgcgggaaagggttgaactgctcgaggggagaatggagatcgaatcagctgagaatcaaggcaccaagatagtgattcatatcccaaccaacgtggaaaagggaaaggagtaa

(19) United States
(12) Patent Application Publication
(10) Pub. No.: US 2024/0368231 A1
(43) Pub. Date: Nov. 7, 2024

(54) IMPROVING CONJUGATION COMPETENCE IN FIRMICUTES

(71) Applicant: BASF SE, Ludwigshafen am Rhein (DE)

(72) Inventors: Tobias MAY, Ludwigshafen (DE); Andrea HEROLD, Ludwigshafen (DE); Daniel Christoph HEINRICH, Ludwigshafen (DE); Heiko SENDROWSKI, Ludwigshafen (DE)

(21) Appl. No.: 18/682,019

(22) PCT Filed: Sep. 9, 2022
(86) PCT No.: PCT/EP22/75128

§ 371 Date: Feb. 7, 2024

(30) Foreign Application Priority Data
Sep. 10, 2021 (EP) 21196073.7

(51) Int. Cl. C07K 14/195; C12N 15/74

(52) U.S. Cl. CPC C07K 14/195; C12N 15/74

(57) ABSTRACT

Described herein are microorganisms, genes, materials and methods for improving genetic competence. In particular, described herein are individual genes/proteins and combinations thereof to improve conjugation competence in Firmicutes, and methods and uses involving such genes, proteins and respective combinations.

The invention is concerned with microorganisms, genes, materials and methods for improving genetic competence. In particular, the invention provides individual genes/proteins and combinations thereof to improve conjugation competence in Firmicutes, and also provides methods and uses involving such genes, proteins and respective combinations.

### BACKGROUND OF THE INVENTION

Firmicutes microorganisms are important microorganisms in industrial fermentation processes. Thus, there is a general need to manipulate the nucleic acids in such microorganisms, for example to render them capable of producing a substance of interest or to prevent or reduce the production of unwanted substances during fermentation. However, the introduction of following nucleic acids is notoriously difficult. Three main mechanisms of nucleic acid transfer have so far been applied: transformation techniques which attempt to directly introduce naked DNA into microorganisms, for example by electroporation; (2) transduction via bacteriophages; (3) conjugation via bacteria. Direct transformation techniques like electroporation have not yielded consistently high efficiencies in Firmicutes, several genera like Paenibacillus, for example, are even generally considered to be non-transformable by common techniques. Transduction via bacteriophages is comparatively cumbersome is and limited in the size of the transfer of the nucleic acid due to the limited bacteriophage capsule volume and the requirement of adding nucleic acid elements required for transfer by the bacteriophagal transfer machinery. Thus, conjugation is considered to be the transfer method of choice for those microorganisms, which have not individually been made transformation competent by exhaustive mutagenesis. In conjugation, a target nucleic acid is provided in a first microorganism (“donor microorganism”) for transfer into a second microorganism (“target microorganism”), wherein the donor microorganism is more amenable to genetic manipulation than the target microorganism. After mixing of the donor and target microorganisms, a plasma bridge is formed between a donor and a target microorganism which allows transfer of linear DNA, plasmid DNA and/or chromosomal bacterial DNA. Even though conjugation techniques overcome many of the sometimes insurmountable obstacles of transformation techniques, transfer efficiency is generally low. Thus, conjugation does not allow efficient transformation of a collection of different target microorganisms. This, however, would be required for example in high throughput environments, for example for the manipulation and subsequent analysis of a library of prospective production hosts. In particular, microorganisms of genus Paenibacillus are in known for their low conjugation efficiency.

It was thus the object of the present invention to provide materials and methods, in particular genes, nucleic acids and proteins, to improve transformability of Firmicutes microorganisms, preferably of genus Paenibacillus.

### SUMMARY OF THE INVENTION

The invention provides a microorganism comprising either
a) a mutant degS gene and optionally a mutant degU gene, or
b) a mutant spo0A gene,
   wherein the microorganism exhibits increased conjugation competence relative to the corresponding wild type strain.

The invention also provides a method of increasing conjugation competence of a microorganism, comprising the step of providing, in the microorganism, either
a) a mutant degS gene, wherein
the degS gene codes for a DegS protein lacking a functional single binding domain, a functional phosphoacceptor domain and/or a functional ATPase domain and/or
the degS gene codes for a DegS protein, wherein the mutation comprises or consists of L99F, L99C, L99D, L99E, L99G, L99H, L99K, L99N, L99P, L99Q, L99R, L99S, L99W or L99Y, and optionally a mutant degU gene, wherein
the degU gene codes for a DegU protein having reduced DNA binding activity and/or lacks a functional DNA binding domain, and/or
   the degU gene codes for a DegU protein, wherein the mutation comprises or consists of, in decreasing order of preference for each alternative aa) and ab), one or more of:
aa) Q218*, Q218K, Q218N, Q218D, Q218R
ab) D223*, D223*+M220N, D223*+M220N+E221G, D223*+M220N+V222G, D223*+M220N+E221G+V222G, D223*+M220D, D223*+M220E, D223*+M220H, D223*+M220F, D223*+M220W, D223*+M220S, D223*+M220A
   or
b) a mutant spo0A gene, wherein
ba) the mutation is located in the DNA binding or receiver domain and results in a reduction or elimination of phosphorylation of the Spo0A protein and/or a reduction or elimination of dimerisation, and/or
bb) the mutation consists of or comprises any of
A257V, more preferably A257S,
I161R, more preferably I161L,
in decreasing order of preference: A257S+I161I, A257A+I161L, A257V+I161I, A257S+I161F or A257A+I161R.

The invention also provides a method of transferring genetic material between two microorganisms, comprising
1) providing, in a first microorganism,
a) a mutant DegS protein, wherein
the DegS protein lacks a functional single binding domain, a functional phosphoacceptor domain and/or a functional ATPase domain and/or
the mutation comprises or consists of L99F, L99C, L99D, L99E, L99G, L99H, L99K, L99N, L99P, L99Q, L99R, L99S, L99W or L99Y,
   and optionally a mutant DegU protein
having reduced DNA binding activity and/or lacks a functional DNA binding domain, and/or
wherein the mutation comprises or consists of, in decreasing order of preference for each alternative aa) and ab), one or more of:
aa) Q218*, Q218K, Q218N, Q218D, Q218R
ab) D223*, D223*+M220N, D223*+M220N+E221G, D223*+M220N+V222G, D223*+M220N+E221G+V222G, D223*+M220D, D223*+M220E, D223*+M220H, D223*+M220F, D223*+M220W, D223*+M220S, D223*+M220A;
   or
b) a mutant Spo0A protein, wherein
ba) the mutation is located in the DNA binding or receiver domain and results in a reduction or elimination of phosphorylation of the Spo0A protein and/or a reduction or elimination of dimerisation,
   and/or
bb) the mutation consists of or comprises any of
A257V, more preferably A257S,
I161R, more preferably I161L,
in decreasing order of preference: A257S+I161I, A257A+I161L, A257V+I161I, A257S+I161F or A257A+I161R,
   and
2) conjugating the first microorganism with a conjugation competent second microorganism, wherein the first microorganism comprises, before step 2, the genetic material to be transferred.

Furthermore, the invention provides an expression vector, comprising an expression cassette for expression of a counterselectable marker, and either
a) a mutant degS gene, wherein
the degS gene codes for a DegS protein lacking a functional single binding domain, a functional phosphoacceptor domain and/or a functional ATPase domain and/or
the degS gene codes for a DegS protein, wherein the mutation comprises or consists of L99F, L99C, L99D, L99E, L99G, L99H, L99K, L99N, L99P, L99Q, L99R, L99S, L99W or L99Y, and optionally a mutant degU gene, wherein
the degU gene codes for a DegU protein having reduced DNA binding activity and/or lacks a functional DNA binding domain, and/or
the degU gene codes for a DegU protein, wherein the mutation comprises or consists of, in decreasing order of preference for each alternative aa) and ab), one or more of:
aa) Q218*, Q218K, Q218N, Q218D, Q218R
ab) D223*, D223*+M220N, D223*+M220N+E221G, D223*+M220N+V222G, D223*+M220N+E221G+V222G, D223*+M220D, D223*+M220E, D223*+M220H, D223*+M220F, D223*+M220W, D223*+M220S, D223*+M220A,
   or
b) a mutant spo0A gene, wherein
ba) the mutation is located in the DNA binding or receiver domain and results in a reduction or elimination of phosphorylation of the Spo0A protein and/or a reduction or elimination of dimerisation,
   and/or
bb) the mutation consists of or comprises any of
A257V, more preferably A257S,
I161R, more preferably I161L,
in decreasing order of preference: A257S+I161I, A257A+I161L, A257V+I161I, A257S+I161F or A257A+I161R.

And the invention provides the use of either
a) a mutant degS gene, wherein
the degS gene codes for a DegS protein lacking a functional single binding domain, a functional phosphoacceptor domain and/or a functional ATPase domain and/or
the degS gene codes for a DegS protein, wherein the mutation comprises or consists of L99F, L99C, L99D, L99E, L99G, L99H, L99K, L99N, L99P, L99Q, L99R, L99S, L99W or L99Y, and optionally a mutant degU gene, wherein
the degU gene codes for a DegU protein having reduced DNA binding activity and/or lacks a functional DNA binding domain, and/or
the degU gene codes for a DegU protein, wherein the mutation comprises or consists of, in decreasing order of preference for each alternative aa) and ab), one or more of:
aa) Q218*, Q218K, Q218N, Q218D, Q218R
ab) D223*, D223*+M220N, D223*+M220N+E221G, D223*+M220N+V222G, D223*+M220N+E221G+V222G, D223*+M220D, D223*+M220E, D223*+M220H, D223*+M220F, D223*+M220W, D223*+M220S, D223*+M220A,
   or
b) a mutant spo0A gene, wherein
ba) the mutation is located in the DNA binding or receiver domain and results in a reduction or elimination of phosphorylation of the Spo0A protein and/or a reduction or elimination of dimerisation,
   and/or
bb) the mutation consists of or comprises any of
A257V, more preferably A257S,
I161R, more preferably I161L,
in decreasing order of preference: A257S+I161I, A257A+I161L, A257V+I161I, A257S+I161F or A257A+I161R
   for increasing conjugation competence of a microorganism selected from any of the taxonomic ranks
of phylum Firmicutes, class Bacilli, Clostridia or Negativicutes,
more preferably of order Bacillales, Clostridiales, Thermoanaerobacterales, Thermosediminibacterales or Selenomonadales,
more preferably of family Bacillaceae, Paenibacillaceae, Pasteuriaceae, Clostridiaceae, Peptococcaceae, Heliobacteriaceae, Syntrophomonadaceae, Thermoanaerobacteraceae, Tepidanaerobacteraceae or Sporomusaceae,
more preferably of genus Alkalibacillus, Bacillus, Geobacillus, Halobacillus, Lysinibacillus, Piscibacillus, Terribacillus, Brevibacillus, Paenibacillus, Thermobacillus, Pasteuria, Clostridium, Desulfotomaculum, Heliobacterium, Pelospora, Pelotomaculum, Caldanaerobacter, Moorella, Thermoanaerobacter, Tepidanaerobacter, Propionispora or Sporomusa,
more preferably of genus Bacillus, Paenibacillus or Clostridium.

### BRIEF DESCRIPTION OF THE DRAWINGS

FIG. 1 shows an evaluation of genetic competence of Paenibacillus polymyxa strains. Genetic competence of the different variants was evaluated by conjugating the cured strains with E. coli S17-1 harboring pCasPP plasmid from Ruetering et. al 2017 (cf. example 1) as the donor strain. The plasmid contains SpCas9 gene expressed under control of constitutive sgsE promoter from Geobacillus stearothermophilus and does not contain any gRNA targeting P. polymyxa genome. To obtain countable colonies, serial dilution was prepared before plating the conjugated strains onto selection LB plate with antibiotics. Colony forming unit from each strain is then normalized to its respective OD600 used for conjugation. Finally, the number of competency fold is calculated relative to the wild type.

FIG. 2 (regarding the DegS protein) shows a sequence alignment of SEQ ID NO. 2 and the sequence according to Uniprot entry A0A074LBY4_PAEPO. Numbers are given according to the position of Uniprot entry A0A074LBY4_PAEPO sequence. The number of asterisks above each amino acid of the A0A074LBY4_PAEPO sequence indicates the degree of conservation, wherein higher number of stars indicate a stronger conservation. Amino acids given below each amino acid of SEQ ID NO. 2 indicate potential substitutions allowable at the respective position, wherein “-” indicates a gap (deletion relative to the A0A074LBY4_PAEPO sequence). The possible substitutions are listed in the order of their respective preference, wherein a more preferred substitution is indicated closer to the respective position in SEQ ID NO. 2.

FIG. 3 (regarding the DegU protein) shows a sequence alignment of SEQ ID No. 1 and the sequence according to Uniprot entry E3EBP5_PAEPS. Numbers are given according to the position of Uniprot entry E3EBP5_PAEPS sequence. The number of asterisks above each amino acid of the E3EBP5_PAEPS sequence indicates the degree of conservation, wherein higher number of stars indicate a stronger conservation. Amino acids given below each amino acid of SEQ ID No. 1 indicate potential substitutions allowable at the respective position, wherein “-” indicates a gap (deletion relative to the E3EBP5_PAEPS sequence). The possible substitutions are listed in the order of their respective preference, wherein a more preferred substitution is indicated closer to the respective position in SEQ ID No. 1.

FIG. 4 (regarding the Spo0A protein) shows a sequence alignment of SEQ ID NO. 3 and the sequence according to Uniprot entry A0A074LZY6_PAEPO. Numbers are given according to the position of Uniprot entry A0A074LZY6_PAEPO sequence. The number of asterisks above each amino acid of the A0A074LZY6_PAEPO sequence indicates the degree of conservation, wherein higher number of stars indicate a stronger conservation. Amino acids given below each amino acid of SEQ ID NO. 3 indicate potential substitutions allowable at the respective position, wherein “-” indicates a gap (deletion relative to the A0A074LZY6_PAEPO sequence). The possible substitutions are listed in the order of their respective preference, wherein a more preferred substitution is indicated closer to the respective position in SEQ ID NO. 3.

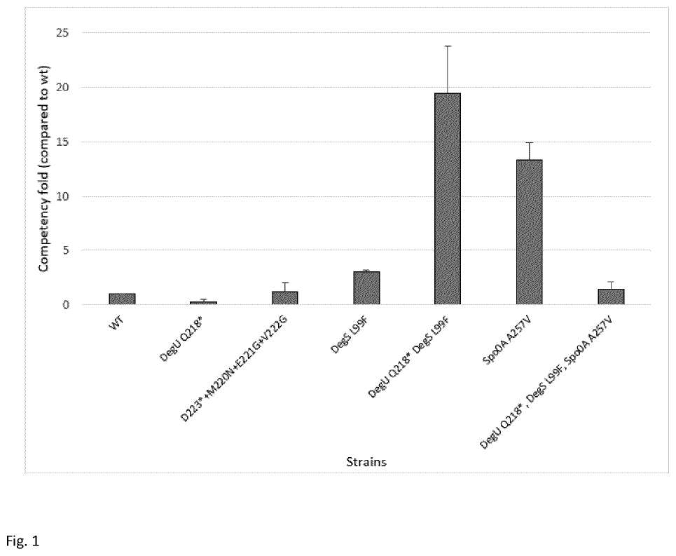

BRIEF DESCRIPTION OF THE SEQUENCES

| SEQ ID | |
|---|---|
| NO. | description |
| 1 | hypothetical DegU protein screening sequence |
| 2 | hypothetical DegS protein screening sequence |
| 3 | hypothetical Spo0A protein screening sequence |
| 4 | artificial sequence for creating a degU Q218* mutation |
| | by changing nucleotide position 652 C to T |
| 5 | artificial sequence for creating a degU “M220N” |
| | (i.e. DegU D223* + M220N + E221G + V222G) |
| | mutation by inserting an A at nucleotide pos. 658 |
| 6 | artificial sequence for creating a degS L99F mutation |
| | by changing nucleotide pos. 295 C to T |
| 7 | artificial sequence for creating a spo0A A257V mutation |
| | by changing nucleotide pos. 770 C to T |
| 8 | nucleotide sequence DSM365 spo0A wild type |
| 9 | nucleotide sequence DSM365 spo0A A257V, pos. 770 C to T |
| 10 | nucleotide sequence DSM365 degU wild type |
| 11 | nucleotide sequence DSM365 degU |
| | D223* + M220N + E221G + V222G, pos. 658 A to AA |
| 12 | nucleotide sequence DSM365 degU Q218*, pos. 652 C to T |
| 13 | nucleotide sequence DSM365 degS wild type |
| 14 | nucleotide sequence DSM365 degS L99F, pos. 295 C to T |

### DETAILED DESCRIPTION OF THE INVENTION

The technical teaching of the invention is expressed herein using the means of language, in particular by use of scientific and technical terms. However, the skilled person understands that the means of language, detailed and precise as they may be, can only approximate the full content of the technical teaching, if only because there are multiple ways of expressing a teaching, each necessarily failing to completely express all conceptual connections, as each expression necessarily must come to an end. With this in mind, the skilled person understands that the subject matter of the invention is the sum of the individual technical concepts signified herein or expressed, necessarily in a pars-pro-toto way, by the innate constrains of a written description. In particular, the skilled person will understand that the signification of individual technical concepts is done herein as an abbreviation of spelling out each possible combination of concepts as far as technically sensible, such that for example the disclosure of three concepts or embodiments A, B and C are a shorthand notation of the concepts A+B, A+C, B+C, A+B+C. In particular, fallback positions for features are described herein in terms of lists of converging alternatives or instantiations. Unless stated otherwise, the invention described herein comprises any combination of such alternatives. The choice of more or less preferred elements from such lists is part of the invention and is due to the skilled person's preference for a minimum degree of realization of the advantage or advantages conveyed by the respective features. Such multiple combined instantiations represent the adequately preferred form(s) of the invention.

In so far as references are made herein to databases entries, e.g., Uniprot entries, the entries are those as published on 2021 May 1 10:00 CET. This also applies to sequences published under the corresponding database entry identifiers.

Nucleic acids and amino acids are abbreviated using their standard one-or three-letter abbreviations. Deletions are indicated by “-”, truncations are indicated by “*”. Alterations of amino acids are specified by the position of the alteration in a respective parent sequence.

As used herein, terms in the singular and the singular forms like “a”, “an” and “the” include plural referents unless the content clearly dictates otherwise. Thus, for example, use of the term “a nucleic acid” optionally includes, as a practical matter, many copies of that nucleic acid molecule; similarly, the term “probe” optionally (and typically) encompasses many similar or identical probe molecules. Also as used herein, the word “comprising” or variations such as “comprises” or “comprising” will be understood to imply the inclusion of a stated element, integer or step, or group of elements, integers or steps, but not the exclusion of any other element, integer or step, or group of elements, integers or steps.

As used herein, the term “and/or” refers to and encompasses any and all possible combinations of one or more of the associated listed items, as well as the lack of combinations when interpreted in the alternative (“or”). The term “comprising” also encompasses the term “consisting of”.

The term “about”, when used in reference to a measurable value, for example an amount of mass, dose, time, temperature, sequence identity and the like, refers to a variation of ±0.1%, 0.25%, 0.5%, 0.75%, 1%, 2%, 3%, 4%, 5%, 6%, 7%, 8%, 9%, 10%, 15% or even 20% of the specified value as well as the specified value. Thus, if a given composition is described as comprising “about 50% X,” it is to be understood that, in some embodiments, the composition comprises 50% X whilst in other embodiments it may comprise anywhere from 40% to 60% X (i.e., 50%±10%).

As used herein, the term “gene” refers to a biochemical information which, when materialised in a nucleic acid, can be transcribed into a gene product, i.e., a further nucleic acid, preferably an RNA, and preferably also can be translated into a peptide or polypeptide. The term is thus also used to indicate the section of a nucleic acid resembling said information and to the sequence of such nucleic acid (herein also termed “gene sequence”).

Also as used herein, the term “allele” refers to a variation of a gene characterized by one or more specific differences in the gene sequence compared to the wild type gene sequence, regardless of the presence of other sequence differences. Alleles or nucleotide sequence variants of the invention have at least, in increasing order of preference, 30%, 40%, 50%, 60%, 70%, 71%, 72%, 73%, 74%, 75%, 76%, 77%, 78%, 79%, 80%, 81%-84%, 85%, 86%, 87%, 88%, 89%, 90%, 91%, 92%, 93%, 94%, 95%, 96%, 97%, 98% or 99% nucleotide “sequence identity” to the nucleotide sequence of the wild type gene. Correspondingly, where an “allele” refers to the biochemical information for expressing a peptide or polypeptide, the respective nucleic acid sequence of the allele has at least, in increasing order of preference, 30%, 40%, 50%, 60%, 70%, 71%, 72%, 73%, 74%, 75%, 76%, 77%, 78%, 79%, 80%, 81%-84%, 85%, 86%, 87%, 88%, 89%, 90%, 91%, 92%, 93%, 94%, 95%, 96%, 97%, 98% or 99% amino acid “sequence identity” to the respective wild type peptide or polypeptide.

Mutations or alterations of amino or nucleic acid sequences can be any of substitutions, deletions or insertions; the terms “mutations” or “alterations” also encompass any combination of these. Hereinafter, all three specific ways of mutating are described in more detail by way of reference to amino acid sequence mutations; the corresponding teaching applies to nucleic acid sequences such that “amino acid” is replaced by “nucleotide”. Mutations can be introduced into the nucleotide sequence of a gene by random or directed mutagenesis techniques. Random mutagenesis techniques include for example UV irradiation and exposition to chemicals, e.g. EMS. Directed mutagenesis techniques include primer extension, meganucleases, zinc finger nucleases and CRISPR-type template directed mutagenesis.

“Substitutions” are described by providing the original amino acid followed by the number of the position within the amino acid sequence, followed by the substituted amino acid. For example, the substitution of histidine at position 120 with alanine is designated as “His120Ala” or “H120A”.

“Deletions” are described by providing the original amino acid followed by the number of the position within the amino acid sequence, followed by “-”. Accordingly, the deletion of glycine at position 150 is designated as ““Gly150-” or “G150-”. Alternatively, deletions are indicated by e.g. “deletion of D183 and G184”.

“Terminations” are described by providing the original amino acid followed by the number of the position within the amino acid sequence, followed by “*”. Accordingly, an amino acid chain termination at position 150 instead of a glycine at this position is designated as “Gly150*” of “G150*”.

“Insertions” are described by providing the original amino acid followed by the number of the position within the amino acid sequence, followed by the original amino acid and the additional amino acid. For example, an insertion at position 180 of lysine next to glycine would be designated as “Gly180GlyLys” or “G180GK”. When more than one amino acid residue is inserted, such as e.g. a Lys and Ala after Gly180 this may be indicated as: Gly180GlyLysAla or G180GKA. In cases where a substitution and an insertion occur at the same position, this may be indicated as S99SD+S99A or in short S99AD. In cases where an amino acid residue identical to the existing amino acid residue is inserted, it is clear that degeneracy in the nomenclature arises. If for example a glycine is inserted after the glycine in the above example this would be indicated by G180GG.

Variants comprising multiple alterations are separated by “+”, e.g., “Arg170Tyr+Gly195Glu” or “R170Y+G195E” representing a substitution of arginine and glycine at positions 170 and 195 with tyrosine and glutamic acid, respectively. Alternatively, multiple alterations may be separated by space or a comma, e.g., R170Y G195E or R170Y, G195E respectively.

Where different alterations can be introduced at a position, the different alterations are separated by a comma, e.g., “Arg170Tyr, Glu” represents a substitution of arginine at position 170 with tyrosine or glutamic acid. Alternatively, different alterations or optional substitutions may be indicated in brackets e.g. Arg170[Tyr, Gly] or Arg170{Tyr, Gly} or in short R170[Y,G] or R170{Y, G}.

A special aspect concerning amino acid substitutions are conservative mutations which often appear to have a minimal effect on protein folding resulting in substantially maintained peptide or polypeptide properties of the respective peptide or polypeptide variant compared to the peptide or polypeptide properties of the parent peptide or polypeptide. Conservative mutations are those where one amino acid is exchanged with a similar amino acid. For determination of %-similarity the following applies, which is also in accordance with the BLOSUM62 matrix, which is one of the most used amino acids similarity matrix for database searching and sequence alignments:
Amino acid A is similar to amino acids S
Amino acid D is similar to amino acids E, N
Amino acid E is similar to amino acids D, K and Q
Amino acid F is similar to amino acids W, Y
Amino acid H is similar to amino acids N, Y
Amino acid I is similar to amino acids L, M and V
Amino acid K is similar to amino acids E, Q and R
Amino acid L is similar to amino acids I, M and V
Amino acid M is similar to amino acids I, L and V
Amino acid N is similar to amino acids D, H and S
Amino acid Q is similar to amino acids E, K and R
Amino acid R is similar to amino acids K and Q
Amino acid S is similar to amino acids A, N and T
Amino acid T is similar to amino acids S
Amino acid V is similar to amino acids I, L and M
Amino acid W is similar to amino acids F and Y
Amino acid Y is similar to amino acids F, H and W

Conservative amino acid substitutions may occur over the full length of the sequence of a polypeptide sequence of a functional protein such as a peptide or polypeptide. Preferably such mutations are not pertaining the functional domains of a peptide or polypeptide.

Protein or nucleic acid variants may be defined by their sequence identity when compared to a parent protein or nucleic acid. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:

Seq A:
AAGATACTG length: 9 bases
Seq B:
GATCTGA length: 7 bases

Hence, the shorter sequence is sequence B.

Producing a pairwise global alignment which is showing both sequences over their complete lengths results in

| | |
|---|---|
| Seq A: | AAGATACTG- |
| | \|\|\| \|\|\| |
| Seq B: | --GAT-CTGA |

The “I” symbol in the alignment indicates identical residues (which means bases for DNA or amino acids for proteins). The number of identical residues is 6.

The “-” symbol in the alignment indicates gaps. The number of gaps introduced by alignment within the sequence B is 1. The number of gaps introduced by alignment at borders of sequence B is 2, and at borders of sequence A is 1.

The alignment length showing the aligned sequences over their complete length is 10.

Producing a pairwise alignment which is showing the shorter sequence over its complete length according to the invention consequently results in:

| | |
|---|---|
| Seq A: | GATACTG- |
| | \|\|\| \|\|\| |
| Seq B: | GAT-CTGA |

Producing a pairwise alignment which is showing sequence A over its complete length according to the invention consequently results in:

| | |
|---|---|
| Seq A: | AAGATACTG |
| | \|\|\| \|\|\| |
| Seq B: | --GAT-CTG |

Producing a pairwise alignment which is showing sequence B over its complete length according to the invention consequently results in:

| | |
|---|---|
| Seq A: | GATACTG- |
| | \|\|\| \|\|\| |
| Seq B: | GAT-CTGA |

The alignment length showing the shorter sequence over its complete length is 8 (one gap is present which is factored in the alignment length of the shorter sequence).

Accordingly, the alignment length showing sequence A over its complete length would be 9 (meaning sequence A is the sequence of the invention), the alignment length showing sequence B over its complete length would be 8 (meaning sequence B is the sequence of the invention).

After aligning the two sequences, in a second step, an identity value shall be determined from the alignment. Therefore, according to the present description the following calculation of percent-identity applies:

%-identity= (identical residues/length of the alignment region which is showing the respective sequence of this invention over its complete length)*100. Thus, sequence identity in relation to comparison of two amino acid sequences according to the invention is calculated by dividing the number of identical residues by the length of the alignment region which is showing the respective sequence of this invention over its complete length. This value is multiplied with 100 to give “%-identity”. According to the example provided above, %-identity is: for sequence A being the sequence of the invention (6/9)*100=66.7%; for sequence B being the sequence of the invention (6/8)*100=75%.

The term “expression cassette” means those constructs in which the nucleic acid sequence encoding an amino acid sequence to be expressed is linked operably to at least one genetic control element which enables or regulates its expression (i.e. transcription and/or translation). The expression may be, for example, stable or transient, constitutive or inducible. Ex-pression cassettes may also comprise the coding regions for two or more polypeptides and lead to the transcription of polycistronic RNAs.

The terms “express,” “expressing,” “expressed” and “expression” refer to expression of a gene product (e.g., a biosynthetic enzyme of a gene of a pathway or reaction defined and described in this application) at a level that the resulting enzyme activity of this protein encoded for, or the pathway or reaction that it refers to allows metabolic flux through this pathway or reaction in the organism in which this gene/pathway is expressed in. The expression can be done by genetic alteration of the microorganism that is used as a starting organism. In some embodiments, a microorganism can be genetically altered (e.g., genetically engineered) to express a gene product at an increased level relative to that produced by the starting microorganism or in a comparable microorganism which has not been altered. Genetic alteration includes, but is not limited to, altering or modifying regulatory sequences or sites associated with expression of a particular gene (e.g. by adding strong promoters, inducible promoters or multiple promoters or by removing regulatory sequences such that expression is constitutive), modifying the chromosomal location of a particular gene, altering nucleic acid sequences adjacent to a particular gene such as a ribosome binding site or transcription terminator, increasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene using routine in the art (including but not limited to use of antisense nucleic acid molecules, for exam-pie, to block expression of repressor proteins).

The terms “overexpress”, “overexpressing”, “overexpressed” and “overexpression” refer to expression of a gene product, in particular to enhancing the expression of a gene product at a level greater than that present prior to a genetic alteration of the starting microorganism. In some embodiments, a microorganism can be genetically altered (e.g., genetically engineered) to express a gene product at an increased level relative to that produced by the starting microorganism. Genetic alteration includes, but is not limited to, altering or modifying regulatory sequences or sites associated with expression of a particular gene (e.g., by adding strong promoters, inducible promoters or multiple promoters or by removing regulatory sequences such that expression is constitutive), modifying the chromosomal location of a particular gene, altering nucleic acid sequences adjacent to a particular gene such as a ribosome binding site or transcription terminator, increasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene using routine in the art (including but not limited to use of antisense nucleic acid molecules, for example, to block expression of repressor proteins). Another way to overexpress a gene product is to enhance the stability of the gene product to increase its life time. The terms “overexpress”, “overexpressing”, “overexpressed” and “overexpression” can also mean that a gene activity is introduced into a microorganism where the respective gene activity, has not been observed before, e.g. by introducing a recombinant gene, e.g. a heterologous gene, in one or more copies into the microorganism preferably by means of genetic engineering.

The invention provides a microorganism which exhibits increased conjugation competence relative to the corresponding wild type strain. This is achieved because the microorganism of the present invention (a) comprises a mutant degS gene and preferably a mutant degU gene but not a mutant spo0A gene, or (b) comprises a mutant spo0A gene without comprising a mutant degS gene and preferably also without comprising a mutant degU gene. This was surprising in view of a publication by Hamoen et al. The pleiotropic response regulator DegU functions as a priming protein in competence development in Bacillus subtilis, PNAS 2000, 9246-9251, which describes that inactivation of the degS-degU operon decreased genetic competence, various inactivation of degS left competence unaffected. Furthermore, Verhamme et al., DegU co-ordinates multicellular behaviour exhibited by Bacillus subtilis, Molecular Microbiology, 2007, 554-568, describe that in Bacillus subtilis genetic competence development is independent of DegS. Furthermore, Spo0A is known to down-regulate AbrB gene expression, which in turn is a repressor of comK expression, which in turn is the key factor of competence development in Bacillus subtilis. Thus, it had to be expected that modifications of the degS and preferably also the degU gene or of the spo0A gene would at best leave competence unaffected or would even decrease competence. Other microorganisms, in particular of genus Paenibacillus, do not even comprise a homolog of the comK gene of Bacillus subtilis; regulation of genetic competence was thus unforeseeable. Finally, publication WO2019221988 in example 3 describes targeting, via conjugation, Paenibacillus strains comprising DegS and/or DegU mutants without any apparent effect on conjugation efficiency. It was thus surprising that conjugation competence could be increased by mutating the genes coding for DegS, DegU and Spo0A, and that this increase only manifests when either the gene coding for Spo0A or a gene coding for DegS or DegU are mutated.

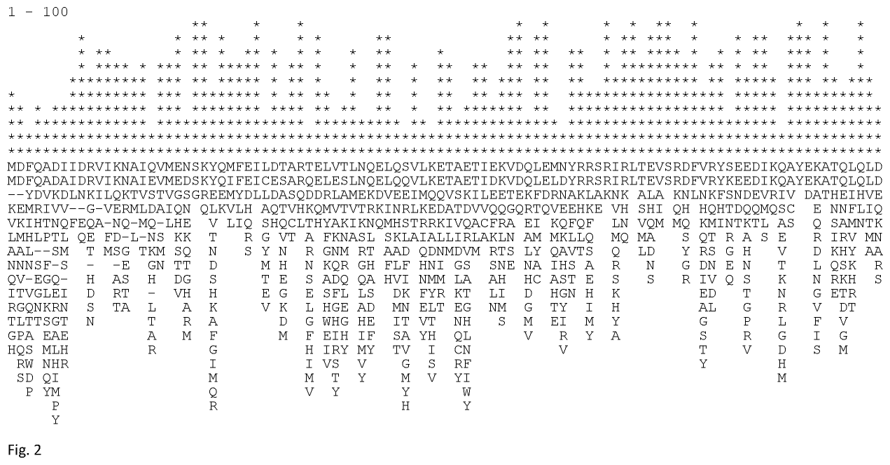

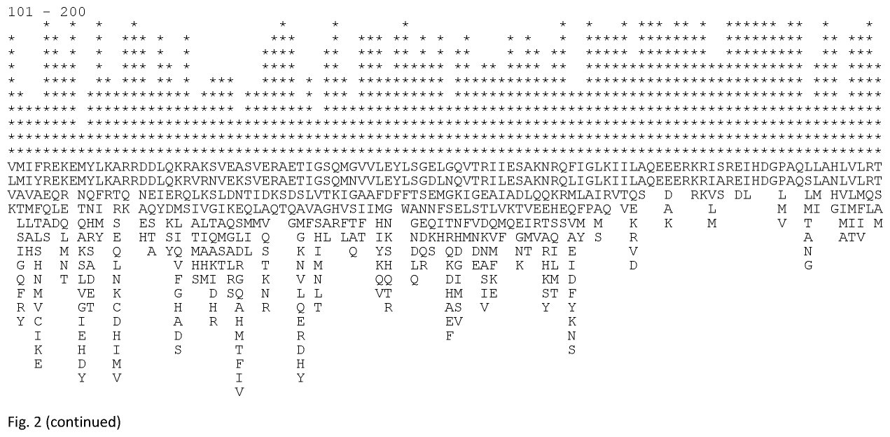

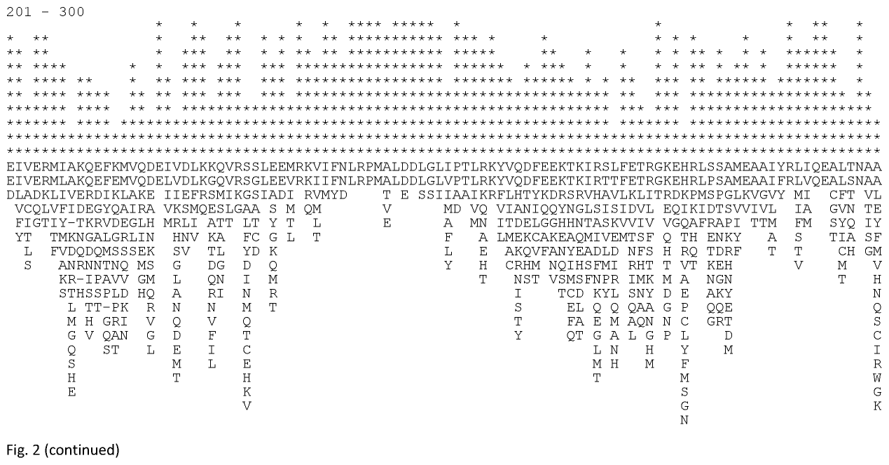

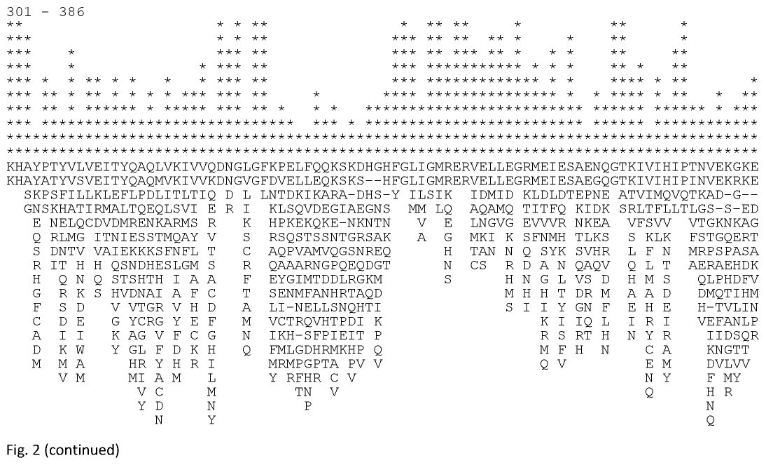

The invention accordingly provides a microorganism comprising a mutant degS gene. The mutant degS gene, when expressed in the microorganism, results in the production of a mutant DegS protein, a degS gene codes for a DegS protein. According to the invention a wild type DegS protein is a member of the DegS type signal transduction histidine kinase family (InterPro ID IPR016381) and comprises, using InterPro notation, a sensor DegS domain (IPR008595) and a histidine kinase domain (IPR005467). According to Pfam nomenclature the wild type DegS protein comprises a sensor protein DegS domain (PF05384), a HisKA_3 histidine kinase domain (PF07730) and a HATPase_c GHKL domain (PF02518). Preferably the wild type degS gene codes for a DegS protein whose amino acid sequence has at least 40%, more preferably at least 43%, more preferably at least 46%, more preferably at least 50%, more preferably at least 58%, more preferably at least 64%, more preferably at least 79%, more preferably at least 84% sequence identity to SEQ ID NO. 2, wherein preferably the sequence identity to SEQ ID NO. 2 is at most 95%, more preferably at most 91%. Particularly preferred the wild type DegS protein has 50-95% sequence identity to SEQ ID NO. 2, more preferably 58-89%. It is to be understood that SEQ ID NO. 2 is an artificial amino acid sequence specifically constructed as a template for amino acid sequence screening and annealing purposes. The sequence can thus be used for identification of degS genes independent from the fact that no DegS activity of the polypeptide of SEQ ID NO. 2 is shown herein. Particularly preferred as a wild type degS gene in a method or plant according to the present invention is any of the amino acid sequences defined by the following Uniprot identifiers, in decreasing order of preference: A0A074LBY4_PAEPO, E3EBP6_PAEPS, A0A4R6MVR0_9BACL, A0A069DLG2_9BACL, A0A268SAI9_9BACL, A0A1X7GB86_9BACL, A0A0M2VLZ1_9BACL, A0A1R1EED0_9BACL, A0A0B0HR83_9BACL, A0A4P8XRM7_9BACL, W7YPT3_9BACL, A0A433XGY7_9BACL, D3EMG1_GEOS4, V9GK22_9BACL, A0A269W177_9BACL, A0A3Q8SA22_9BACL, A0A1E3L2X6_9BACL, A0A369BCF2_9BACL, A0A2S0UEJ3_9BACL, A0A090XSK7_PAEMA, A0A3S1DMQ5_9BACL, A0A1B1N3X4_9BACL, C6J2I3_9BACL, A0A172ZLS7_9BACL, A0A1G7PLD1_9BACL, A0A2Z2KM36_9BACL, A0A3Q9IDP6_9BACL, A0A0D3VFE7_9BACL, A0A168QEJ2_9BACL, A0A1B8VU57_9BACI, W4EHN2_9BACL, A0A0E4CZI9_9BACL, A0A089L4Y0_9BACL, A0A098MEC1_9BACL, A0A089IPW0_9BACL, 0A167D837_9BACL, A0A089NAN3_9BACL, X4ZSE0_9BACL, A0A2W1M2J1_9BACL, A0A1I0JTZ3_9BACL, A0A4Q2LW12_9BACL, A0A1I3PZ40_9BACL, A0A401I4T4_9BACL, A0A1B8UU84_9BACL, A0A1T2X709_9BACL, A0A2N5NDJ2_9BACL, A0A0F5RAF5_9BACL, A0A3G9IYB3_9BACL, A0A1H1WM07_9BACL, H3S9W1_9BACL, A0A1I6WIJ5_9BACL, A0A0D5NQK3_9BACL, A0A015KKW1_9BACL, A0A3D9SD21_9BACL, A0A1B8VZZ2_9BACI, M9LFD8_PAEPP, A0A2V4WBE9_9BACL, A0A0U2WI25_9BACL, A0A1R1DAI7_9BACL, A0A368VS81_9BACL, E0IEE5_9BACL, A0A371P0S2_9BACL, A0A4P6EY40_9BACL, L0EJK6_THECK, A0A3D9I772_9BACL, A0A1X7KY93_9BACL, A0A231R9F5_9BACL, A0A3A1UXN0_9BACL, A0A494XFI9_9BACL, A0A1Y5KD15_9BACL, A0A398CI63_9BACL, A0A3T1DDD1_9BACL, A0A0Q4RDM1_9BACL, C6D5A2_PAESJ, A0A2V2Z262_9BACL, A0A3G3K194_9BACL, A0A3D9KBV6_9BACL, A0A1A5YD71_9BACL, A0A433R8L8_9BACL, A0A172TIT6_9BACL, A0A1I1BCS1_9BACL, A0A081P315_9BACL, A0A4R5KGY1_9BACL, A0A229USJ3_9BACL, A0A2V5JWK3_9BACL, A0A329MCI0_9BACL, A0A0U2INE8_9BACL, A0A1K1QX93_9BACL, A0A2W1NWZ0_9BACL, A0A1I4LCQ8_9BACL, A0A329L6X4_9BACL, A0A0C2V9A2_9BACL, A0A4Q9DIC7_9BACL, H6NT63_9BACL, A0A1H4RQL7_9BACL, A0A3B0C2F8_9BACL, V9VZ78_9BACL, A0A1H0L1L7_9BACL, A0A430JA16_9BACL, F5LST9_9BACL, A0A4R4EAF4_9BACL, A0A0Q7JRA6_9BACL, A0A1V4HGJ0_9BACL, A0A1C0ZYJ1_9BACL, A0A4R3KJJ5_9BACI, M8DFK7_9BACL, A0A1U9KAR4_9BACL, A0A3M8DWV1_9BACL, A0A1A5XKA3_9BACL, A0A1E5LA89_9BACL, A0A074LTT3_9BACL, A0A1I4CE81_9BACL, C0Z731_BREBN, V6MBX1_9BACL, A0A1Y0IJ16_9BACL, A0A3M8BE71_9BACL, A0A4Q1STZ5_9BACL, A0A1E5G3N9_9BACL, A0A075RB77_BRELA, A0A419SF93_9BACL, A0A1Z5HTH4_9THEO, F5L9B1_CALTT, A0A3S9T1P5_9FIRM, A0A2N5M9N1_9BACI, A0A235FGA1_9BACI, A0A0M2U6G0_9FIRM, A0A4R6TU87_9BACI, A0A1E5LDM8_9BACI, A0A498RIM9_9FIRM, 0A120HRZ3_9BACL, A0A4Q0VV23_9BACI, A0A1I2EJ29_9BACI, A0A1U7MGK1_9FIRM, E6TSA5_BACCJ, A0A3E2JMS2_9BACI, A0A1I4L1V6_9BACI, A0A2P8HQR2_9BACI, Q9K6U6_BACHD, A0A402BS91_9FIRM, A0A4R3MVB0_9BACI, X0RMB4_9BACI, A0A4R2RM72_9FIRM, A0A1H4AT83_9BACI, A0A292YC86_9BACL, C5D873_GEOSW, Q5KV50_GEOKA, 0A1W2BFW9_9FIRM, A0A285CZH1_9BACI and A0A1G9QKZ2_9FIRM. Particularly preferred according to the invention are wild type DegS protein sequences and corresponding degS genes coding therefor which have at least 40%, more preferably at least 46%, more preferably at least 58% and even more preferably 80-100% sequence identity to the amino acid sequence given by Uniprot identifier A0A074LBY4_PAEPO. When not considering the specific mutations to the DegS protein sequence described according to the invention, the mutant DegS protein preferably differs from the amino acid sequence given by Uniprot identifier A0A074LBY4_PAEPO by 0-40 amino acids, more preferably 0-20 amino acids, even more preferably 0-10 amino acids, even more preferably 1-5 amino acids, wherein those differences preferably conform to the constraints according to FIG. 2. If the mutant DegS sequence, when aligned to the sequence according to Uniprot identifier A0A074LBY4_PAEPO, is longer than said sequence, then each C- or N-terminal extension is preferably no longer than 10 amino acids, more preferably 0-5 amino acids.

The invention furthermore provides a microorganism comprising a mutant degU gene in addition to the mutant degS gene. The mutant degU gene, when expressed in the microorganism, results in the production of a mutant DegU protein, a degU gene codes for a DegU protein. According to the invention a wild type DegU protein is a member of the CheY-like superfamily (InterPro ID IPR011006) and comprises, using InterPro notation, a signal transduction response regulator (receiver domain) (IPR001789) and a transcription regulator LuxR domain (C-terminal) (IPR000792). According to Pfam nomenclature the wild type DegU protein comprises a response regulator receiver domain (PF00072, Pao et al., J Mol Evol 1995, 136-154 Response regulators of bacterial signal transduction systems: selective domain shuffling during evolution), and a LuxR-type DNA-binding HTH domain (PF00196).

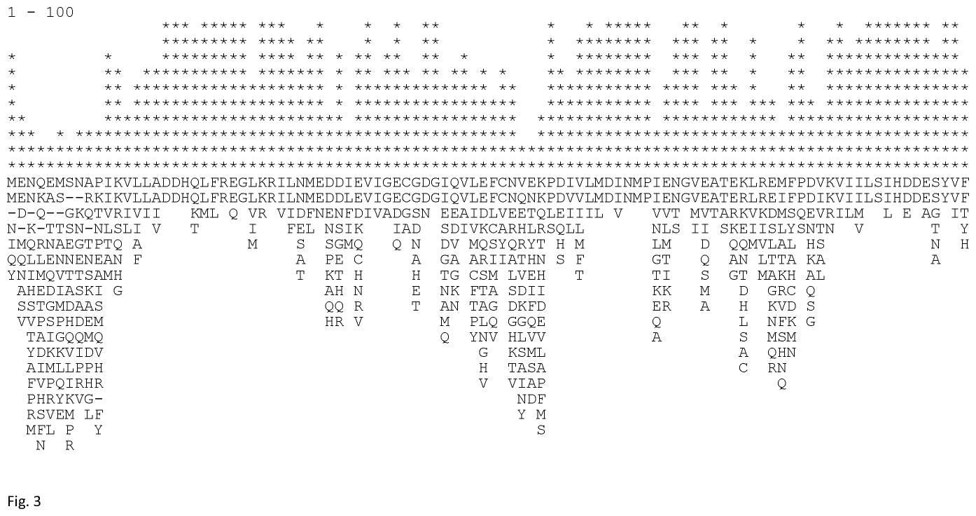

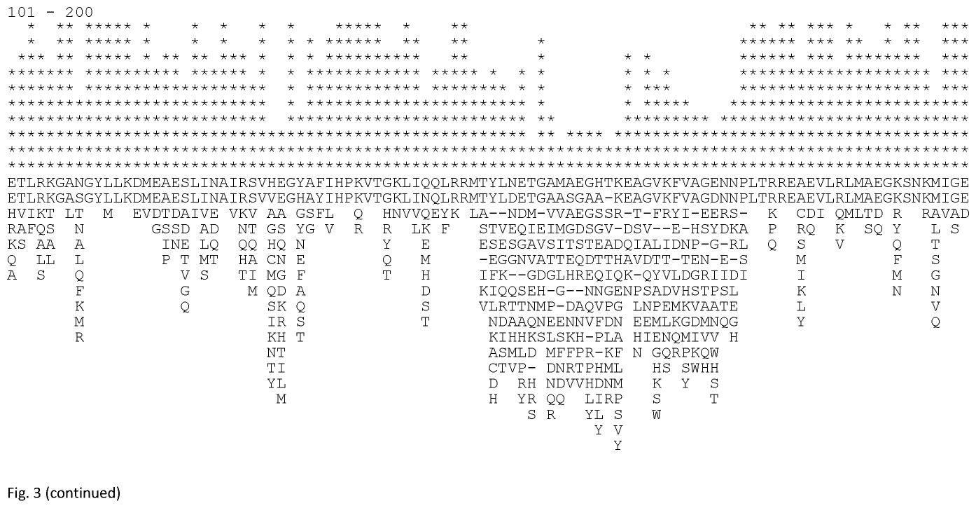

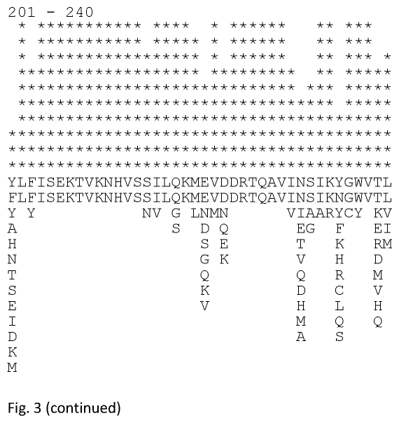

Preferably the wild type degU gene codes for a DegU protein whose amino acid sequence has at least 40%, more preferably at least 43%, more preferably at least 45%, more preferably at least 53%, more preferably at least 57%, more preferably at least 70%, more preferably at least 77%, more preferably at least 85%, more preferably at least 88% sequence identity to SEQ ID No. 1, wherein preferably the sequence identity to SEQ ID No. 1 is at most 95%, more preferably at most 92%. Particularly preferred the wild type DegU protein has 50-95% sequence identity to SEQ ID No. 1, more preferably 77-91%. It is to be understood that SEQ ID No. 1 is an artificial amino acid sequence specifically constructed as a template for amino acid sequence screening and annealing purposes. The sequence can thus be used for identification of degU genes independent from the fact that no DegU activity of the polypeptide of SEQ ID No. 1 is shown herein. Particularly preferred as a wild type DegU gene in a method or plant according to the present invention is any of the amino acid sequences defined by the following Uniprot identifiers, in decreasing order of preference: E3EBP5_PAEPS, A0A4R6MUX9_9BACL, A0A268SA79_9BACL, A0A069DEZ2_9BACL, A0A0B0HVN5_9BACL, W4EI28_9BACL, A0A1X7GB62_9BACL, A0A089MEU3_9BACL, A0A0E4HEC8_9BACL, A0A4P8XUS1_9BACL, A0A0M2VKR6_9BACL, A0A089M364_9BACL, V9GIW8_9BACL, W7YTM0_9BACL, A0A098MFT1_9BACL, D3EMG0_GEOS4, A0A1B8VU54_9BACI, A0A2Z2KSF3_9BACL, A0A269W3P3_9BACL, A0A1R1EEL5_9BACL, X5A6E5_9BACL, A0A089IT67_9BACL, A0A1I0JV80_9BACL, A0A168QEL2_9BACL, A0A0D3VFM7_9BACL, A0A172ZLN3_9BACL, A0A167D848_9BACL, A0A1E3L0K1_9BACL, A0A2W1LCB1_9BACL, A0A0U2N3N5_9BACL, L0EHW2_THECK, A0A1T2X729_9BACL, A0A1B8UUC0_9BACL, H3S9W0_9BACL, A0A3D9SC72_9BACL, A0A401I4R6_9BACL, A0A1I6WHZ6_9BACL, A0A015NM30_9BACL, A0A0F5R725_9BACL, A0A2N5NDN0_9BACL, M9LLL0_PAEPP, A0A0D5NRR7_9BACL, A0A2S0UEL3_9BACL, A0A4Q2M1I7_9BACL, A0A1H1WMP7_9BACL, A0A3A1US45_9BACL, A0A3G9JII4_9BACL, C6D5A1_PAESJ, A0A433XGQ7_9BACL, A0A1I3PZK5_9BACL, A0A1R1DAD8_9BACL, A0A4P6F1N4_9BACL, A0A0Q4R517_9BACL, A0A172TIH8_9BACL, A0A2V4X724_9BACL, A0A1Y5KD60_9BACL, A0A368VSS2_9BACL, A0A1B8VZY5_9BACI, A0A371P0X4_9BACL, A0A231RB89_9BACL, A0A369BC27_9BACL, E0IEE4_9BACL, A0A2V2YZQ8_9BACL, A0A1G7PNR5_9BACL, A0A3S1BJF8_9BACL, A0A1A5YDL9_9BACL, A0A0U2WGN9_9BACL, A0A494X986_9BACL, A0A3D9KD00_9BACL, C6J2I4_9BACL, A0A3Q9IF25_9BACL, A0A3G3K27_9BACL, A0A090XUD0_PAEMA, A0A3D9I787_9BACL, A0A398CFS8_9BACL, A0A1B1N3Y9_9BACL, A0A081P3I6_9BACL, A0A3T1DDG1_9BACL, A0A1K1QXK1_9BACL, A0A3Q8SA76_9BACL, A0A1X7KWC6_9BACL, A0A229USY4_9BACL, A0A4Q9DKY4_9BACL, A0A4R5KE70_9BACL, A0A329L4V8_9BACL, A0A2W1N4T3_9BACL, A0A1I1BB61_9BACL, H6NT64_9BACL, A0A1I4LD00_9BACL, A0A329MBB6_9BACL, A0A3S1AKF3_9BACL, F5LST8_9BACL, A0A1V4HGJ1_9BACL, A0A1H0L0T9_9BACL, A0A0Q7JPS4_9BACL, A0A1H4RQ86_9BACL, A0A3S0BTA6_9BACL, A0A1C0ZYC4_9BACL, A0A0C2RFX7_9BACL, V9W4A0_9BACL, A0A2V5KBB6_9BACL, A0A3B0C3G8_9BACL, A0A4R4EFH3_9BACL, A0A1U9KAL0_9BACL, A0A4R3KIF8_9BACI, A0A292YJB9_9BACL, A0A075RHH4_BRELA, A0A0D1XDF4_ANEMI, A0A1A5XJS0_9BACL, V6M9Z2_9BACL, A0A120HRZ5_9BACL, A0A419V950_9BACL, A0A3R9QNM1_9BACI, A0A1I4L117_9BACI, A0A1H0J2F9_9BACI, A0A3M8DYQ5_9BACL, A0A1I2EIB9_9BACI, A0A428N9S8_9BACI, A0A2P6MHC1_9BACI, A0A1I4CG56_9BACL, C0Z730_BREBN, M8DFP6_9BACL, A0A345BZD8_9BACI, A0A419SF78_9BACL, A0A3M8BE38_9BACL, A0A1H9W953_9BACI, A0A4Q1ST01_9BACL, F5L9B2_CALTT, A0A1G8AEE7_9BACI, D6Y0E8_BACIE, A0A4Q0VW28_9BACI, A0A2T4U7P0_9BACI, A0A061NX68_9BACL, A0A061P3R3_9BACL, A0A098EIU7_9BACL, A0A3M8P3C8_9BACL, A0A1H2UAM2_9BACI, A0A3A9KCQ9_9BACI, A0A1Y0IJ22_9BACL, A0A1G8E1Q8_9BACI, A0A1S2M8P6_9BACI, Q9K6U7_BACHD, A0A4R3N1F6_9BACI, A0A437KCI7_9BACI, A0A2P8GCB9_9BACL, A0A1X9MFG9_9BACI, A0A1H9TTG1_9BACI, A0A327YHU2_9BACI and A0A368Y3Q5_9BACI. Particularly preferred according to the invention are wild type DegU protein sequences and corresponding degU genes coding therefor which have at least 45%, more preferably at least 51%, more preferably at least 54% and even more preferably 73-100% sequence identity to the amino acid sequence given by Uniprot identifier E3EBP5_PAEPS. When not considering the specific mutations to the DegU protein sequence described according to the invention, the mutant DegU protein preferably differs from the amino acid sequence given by Uniprot identifier E3EBP5_PAEPS by 0-20 amino acids, more preferably 0-15 amino acids, even more preferably 0-10 amino acids, even more preferably 1-5 amino acids, wherein those differences preferably conform to the constraints according to FIG. 3. If the mutant DegU sequence, when aligned to the sequence according to Uniprot identifier E3EBP5_PAEPS, is longer than said sequence, then each C- or N-terminal extension is preferably no longer than 10 amino acids, more preferably 0-5 amino acids.

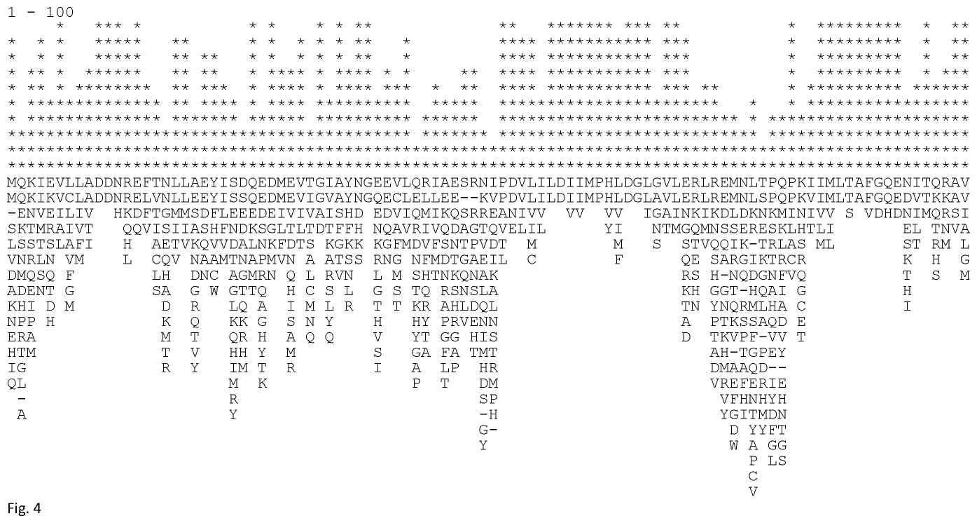

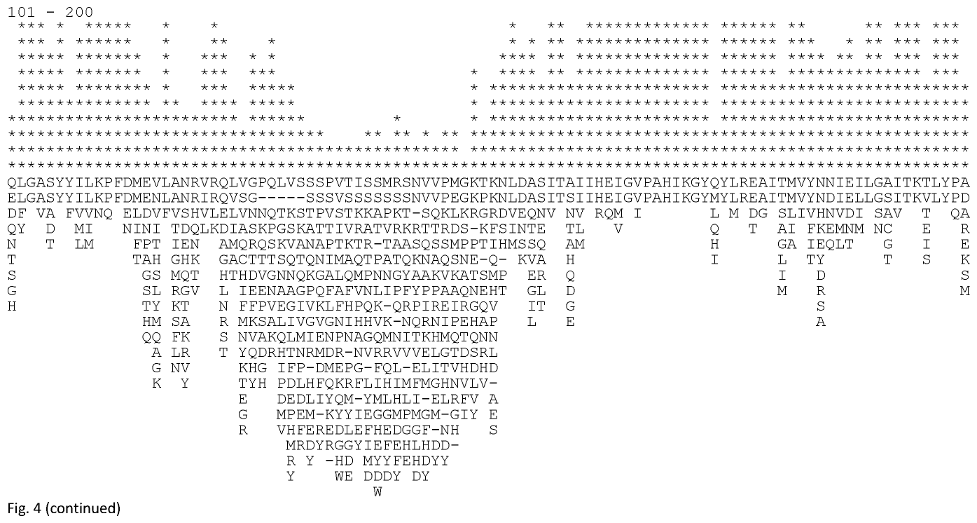

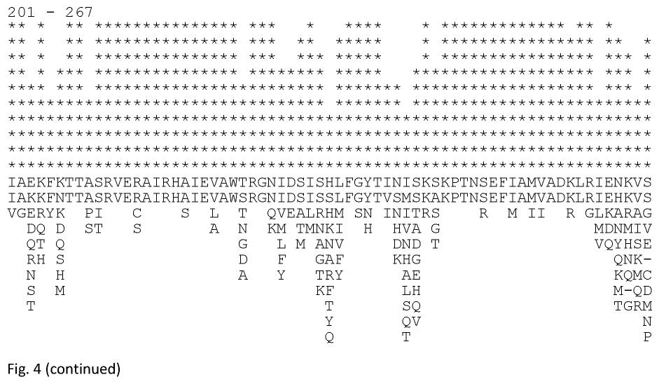

According to the invention, the microorganism can comprise a mutant spo0A gene. The mutant spo0A gene, when expressed in the microorganism, results in the production of a mutant Spo0A protein, a spo0A gene codes for a Spo0A protein. According to the invention a wild type Spo0A protein is a member of the Sporulation transcription factor Spo0A (IPR012052) and comprises, using InterPro notation, a Signal transduction response regulator receiver domain (IPR001789) and a Sporulation initiation factor Spo0A C-terminal domain (IPR014879), which is part of a Winged helix-like DNA-binding domain superfamily (IPR036388). According to Pfam nomenclature the wild type Spo0A protein comprises a Response regulator receiver domain (PF00072) and a Sporulation initiation factor Spo0A C terminal domain (PF08769). Preferably the wild type spo0A gene codes for a Spo0A protein whose amino acid sequence has at least 45%, more preferably at least 56%, more preferably at least 69%, more preferably at least 70%, more preferably at least 67%, more preferably at least 70%, more preferably at least 73%, more preferably at least 74%, more preferably 75% sequence identity to SEQ ID NO. 3, wherein preferably the sequence identity to SEQ ID NO. 3 is at most 85%, more preferably at most 11%. Particularly preferred the wild type Spo0A protein has 50-85% sequence identity to SEQ ID NO. 3, more preferably 76-84%. It is to be understood that SEQ ID NO. 3 is an artificial amino acid sequence specifically constructed as a template for amino acid sequence screening and annealing purposes. The sequence can thus be used for identification of Spo0A genes independent from the fact that no Spo0A activity of the polypeptide of SEQ ID NO. 3 is shown herein. Particularly preferred as a wild type Spo0A gene in a method or plant according to the present invention is any of the amino acid sequences defined by the following Uniprot identifiers, in decreasing order of preference: A0A074LZY6_PAEPO, E0RDX7_PAEP6, H6CM41_9BACL, A0A0D7WZ78_9BACL, A0A167DI09_9BACL, W7YKB3_9BACL, A0A168BRF7_9BACL, A0A1G5JWJ2_9BACL, A0A168P4Q5_9BACL, A0A168M3D7_9BACL, A0A1R1EUX4_9BACL, A0A2W6PE29_9BACL, A0A2V4WTN3_PAEBA, A0A328WGM0_PAELA, D3E6N2_GEOS4, G4HF05_9BACL, A0A1R0XBX0_9BACL, A0A098M8U8_9BACL, A0A3Q8SBT8_9BACL, A0A0M1P3N3_9BACL, R9LQX4_9BACL, A0A2Z2KRN4_9BACL, A0A1B8WQN2_9BACI, A0A089MEU2_9BACL, A0A089LZP7_9BACL, A0A0F7 FA95_PAEDU, A0A0E4HDK7_9BACL, A0A1G7R7Q0_9BACL, A0A1H8N6P6_9BACL, X4ZFA8_9BACL, A0A3G9IQE6_9BACL, A0A369BNP1_9BACL, A0A1B1N0I3_9BACL, A0A015KRJ2_9BACL, A0A2N5N5F6_9BACL, A0A1T2XNU8_9BACL, A0A090ZFJ2_PAEMA, A0A3D9QX06_9BACL, E0ICH6_9BACL, A0A1G9E5Z0_9BACL, A0A3S1DUM5_9BACL, A0A0D5NPL4_9BACL, A0A368WCL4_9BACL, A0A4Q2LM98_9BACL, A0A328U1G0_9BACL, A0A172TM01_9BACL, A0A1I2EKA8_9BACL, A0A1A5YCA7_9BACL, A0A371PM84_9BACL, A0A3A6PB13_9BACL, A0A2V2YXM7_9BACL, L0EEN3_THECK, A0A3A1UXY9_9BACL, A0A3B0CH88_9BACL, A0A1V4HR00_9BACL, A0A1V0UWJ6_9BACL, H3SFG5_9BACL, A0A1X7JKH5_9BACL, A0A1I2FDS6_9BACL, A0A3D9IJB3_9BACL, A0A398CE46_9BACL, M9LB51_PAEPP, A0A3D9KSR7_9BACL, A0A081NWT7_9BACL, H6NL94_9BACL, A0A1C0ZWF8_9BACL, A0A4Y8M823_9BACL, A0A1X7HJ70_9BACL, A0A329MFB7_9BACL, A0A1G4P4T7_9BACL, A0A229UXF4_9BACL, A0A0U2WBQ5_9BACL, A0A3S0BM74_9BACL, K4ZP76_PAEA2, A0A2W1NBK6_9BACL, A0A172ZK56_9BACL, A0A3M8CIR1_9BACL, M8EE17_9BACL, A0A0Q3T5E2_BRECH, A0A0K9YRB7_9BACL, A0A1I3U483_9BACL, V6MCA1_9BACL, C0ZC17_BREBN, A0A4R3KM88_9BACI, A0A3M8B6E4_9BACL, A0A2N3LN87_9BACI, A0A419SMW9_9BACL, A0A3M8D088_9BACL, A0A075R4A3_BRELA, U1X7N0_ANEAE, A0A1H2UFN8_9BACL, A0A0D1VW72_ANEMI, A0A0X8D3E6_9BACL, A0A0U5AZK5_9BACL, A0A4R3L002_9BACL, A0A0Q3WXA1_9BACI, A0A0B0IAE5_9BACI, A0A223KSV6_9BACI, W4PXN5_9BACI, A0A235BCM6_9BACL, A0A235FAK4_9BACI, A0A2T4Z9J8_9BACL, A0A1S2MEZ1_9BACI, Q9K977_BACHD, A0A1S2LUZ3_9BACI, A0A1U9KC16_9BACL, A7Z6J0_BACVZ, Q65HJ7_BACLD, W4QWX1_BACA3, A0A1I6TUX2_9BACL, A0A1I2L3I1_9BACL, A0A0H3E179_BACA1, SP0A_BACSU, A8FF06_BACP2, A0A0J6EVC7_9BACI, A0A417YV34_9BACI, D5DS62_BACMQ, A0A4Q0VQU7_9BACI, A0A1H9PKN5_9BACI, A0A1I3QAI8_9BACL, A0A1G6Q9T8_9BACL, W1SHY1_9BACI, A0A364K8M0_9BACL, A0A150F6K4_9BACI, M5PEN8_9BACI, A0A1S2M754_9BACI, A0A0A8X8S8_9BACI, A0A1R1RU53_9BACI, A0A1S2LYV1_9BACI, A0A1B3XQX6_9BACI, A0A1H8EQX3_9BACL, A0A2N5GRE3_9BACI, A0A4R1B005_9BACI, A0A4R1QFH8_9BACI, A0A1B1Z5W5_9BACI, K6BXH2_9BACI, A0A160F753_9BACI, U5LDF9_9BACI, A0A0M0KYT7_9BACI, A0A061NL57_9BACL, A0A3A1QZJ5_9BACI, A0A2N5H854_9BACI, A0A160ISE8_9BACI, A0A217SRN1_LACSH, A0A1M4TLQ2_9BACL, A0A4R2QSJ5_9BACL, A0A3L7K5H6_9BACI, A0A2N5M452_9BACI, W4QKM5_9BACI, A0A4R2PAA5_9BACL, A0A0J1IMN1_BACCI, R9C857_9BACI, A0A0M4FX23_9BACI, A0A165XSR5_9BACI, A0A179SV99_9BACI, A0A1Y0IS88_9BACL, A0A248TLE9_9BACI, A0A1H0WI33_9BACI, A0A0H4PIL5_9BACI, I8AMT2_9BACI, A0A0D6ZAA3_9BACI, A0A3T0I1Q1_9BACI, A0A1I0SQQ4_9BACI, I3EAA8_BACMM, A0A0M0GB29_SPOGL, A0A1L8ZLZ8_9BACI, A0A370GBM9_9BACI, A0A433H928_9BACI, A0A4R6U795_9BACI, A0A060LXS4_9BACI, A0A074LME5_9BACL, A0A0K9GWU6_9BACI, A0A150KM63_9BACI, K6CV08_BACAZ, A0A323TXM3_9BACI, A0A2N0Z9Q2_9BACI, J8AK67_BACCE, A0A073KUP4_9BACI, A0A292YQZ8_9BACL, A0A226QLR6_9BACI, A0A160FBJ9_9BACI, C3BPR4_9BACI, E6TXR1_BACCJ, A0A1L3MQ53_9BACI, A0A0C2YCQ6_BACBA, Q8EQ49_OCEIH, A0A316D8M3_9BACL, A0A0J6FU61_9BACI, A0A1H8C0C3_9BACI, A0A084J373_BACMY, A0A1I4JPZ3_9BACI, A0A0M2SG37_9BACI, A0A150MMS1_9BACI, A0A1J6WGW3_9BACI, A0A0P6W2Q8_9BACI, A0A1I0SZE6_9BACI, A7GSJ0_BACCN, A0A2C9Z3P6_BACHU, A0A398BG15_9BACI, A0A0V8JFI7_9BACI, A0A1I5NPW8_9BACI, A0A4R2B866_9BACI, A0A023DE04_9BACI, A0A023CLR0_9BACI, A0A327YN47_9BACI, A0A0Q9XV74_9BACI, A0A147K7R5_9BACI, A0A443J408_9BACI, A0A498DDK1_9BACI, A0A0K6GMP9_9BACI, A0A429XD58_9BACI, A0A1I1ZLD5_9BACI, A4IQR2_GEOTN, A0A073K3V0_9BACI, A0A1X7D063_9BACI, Q5WF68_BACSK, A0A3S4RLT9_9BACI, A0A150JT68_BACCO, F5L3H6_CALTT, A0A0M0GPU2_9BACI, S5Z7C0_BACPJ, A0A1Z2V3H9_9BACI, A0A3A9KGU4_9BACI, A0A285CLU9_9BACI, A0A366XYH1_9BACI, A0A0D8BRF6_GEOKU, A0A265NFG5_9BACI, A0A428N868_9BACI, A0A2P8HAG1_9BACI, A0A1H0B3U0_9BACI, A0A150M7C5_9BACI, A0A1G8D1C3_9BACI, A0A1G8BRD8_9BACI, A0A4Q4IIH6_9BACL, A0A4Y9AEG4_9BACI, A0A1I0FQG9_9BACI, A0A0F5HWK7_9BACI, A0A1H1BJ69_9BACI, W9A8X3_9BACI, A0A1H9LW77_9BACI, A0A494Z0K1_9BACI, A0A1M5CXL0_9BACI, A0A1G8JJN9_9BACI, A0A1G6IGH8_9BACI, A0A4Y7S8L6_9FIRM, A0A1X9MFG7_9BACI, A0A0A2UZF1_9BACI, A0A1H9ZLP9_9BACI, A0A1M4XLK4_9CLOT, A0A0A5GIF6_9BACI, A0A0C2VIM1_9BACL, A0A2A2IDA3_9BACI, A0A366EJ45_9BACI, A0A317KZA9_9BACI, A0A0A5GEQ9_9BACI, A0A1E5LK88_9BACI, A0A2S5GEL8_9BACL, A0A1G9LM94_9BACI, A0A1N6PFX1_9BACI, A0A1E7DMX2_9BACI, N4WSS3_9BACI, A0A1I1T1Z9_9BACI, A0A0A1MZ98_9BACI, A0A4R3N0Q4_9BACI, A0A4Y8KST9_9BACL, A0A2U1K6N5_9BACI, A0A075LLD7_9BACI, A0A1I0V6R2_9BACI, A0A0U1KL95_9BACI, A0A2P6MK99_9BACI, C8WXF8_ALIAD, A0A1M6KIQ3_9CLOT, A0A1L8CTW3_9THEO, A0A1V2A9Q9_9BACI, A0A2T4UAN8_9BACI, A0A4Z0GKV9_9BACL, A0A1M6I6U1_9FIRM, A0A1I2VPT9_9BACL, A0A1M6S6D3_9BACL, A0A1N7KMH0_9BACL, A0A140L8E0_9CLOT, A0A090J299_9BACI, V6IWU0_9BACL, A0A024P5H3_9BACI, A0A285NM88_9BACI, A0A143MRA0_9BACI, A0A0A5GCA3_9BACI, A0A0U1QSI9_9BACL, A0A0B5AS70_9BACL, A0A1M6C4X1_9CLOT, A0A2I0QX97_9BACI, A0A0P9EJT7_9BACL, A0A1U7MLA0_9FIRM, A0A2T0BRS8_9CLOT, A0A1H2T3C9_9BACL, A0A1H9A7M5_9BACI, A0A084JIX0_9CLOT, D9SLV6_CLOC7, A0A4R2RWP5_9FIRM, U2CLF1_9FIRM and A0A1G8VG90_9BACI. Particularly preferred according to the invention are wild type Spo0A protein sequences and corresponding spo0A genes coding therefor which have at least 55%, more preferably at least 60%, more preferably at least 62%, more preferably at least 70%, even more preferably 80-100% and even more preferably 95-100% sequence identity to the amino acid sequence given by Uniprot identifier A0A074LZY6_PAEPO. When not considering the specific mutations to the Spo0A protein sequence described according to the invention, the mutant Spo0A protein preferably differs from the amino acid sequence given by Uniprot identifier A0A074LZY6_PAEPO by 0-20 amino acids, more preferably 0-15 amino acids, even more preferably 0-10 amino acids, even more preferably 1-5 amino acids, wherein those differences preferably conform to the constraints according to FIG. 4. If the mutant Spo0A sequence, when aligned to the sequence according to Uniprot identifier A0A074LZY6_PAEPO, is longer than said sequence, then each C- or N-terminal extension is preferably no longer than 30 amino acids, more preferably 0-10 amino acids.

It is a particular advantage of the present invention that the invention allows to increase conjugation competence by mutation of one or two genes ubiquitously found in Firmicutes microorganisms. Thus, the teaching of the present invention is applicable not only to microorganisms of genus Paenibacillus as shown in the examples below, but can also be used to increase conjugation efficiency for other Firmicutes. Preferred microorganisms are described below.

It is a further advantage that the DegS, DegU and DegS+DegU mutants of the present invention do not abolish or significantly reduce the microorganism's capability of sporulation. This is a particular advantage for spore forming plant health compositions or other applications which rely on spore formation.

The DegS protein preferably lacks a functional single binding domain, a functional phosphoacceptor domain and/or a functional ATPase domain. The presence of these traits can be easily identified in the microorganism of the present invention, preferably of genus Paenibacillus, by observing an increase conjugation competence compared to the corresponding wild type strain, and can be easily achieved, for example by introducing a mutation in the sensor DegS domain (IPR008595).

As described above, the wild type DegS protein comprises a Sensor DegS domain; this domain extends, according to the numbering of the protein sequence with Uniprot identifier A0A074LBY4_PAEPO, from amino acid position 10 to 165. Further information on the DNA binding domain is available from the corresponding Pfam and InterPro databases. For example, for the most preferred wild type DegS protein sequence A0A074LBY4_PAEPO the DNA-binding domain is predicted to comprise 2 alpha-helix domains, spanning the positions 5-81 and 84-186, wherein the amino acids of positions 175-186 already overlap with the histidine kinase domain. It is preferred if the DegS protein DNA-binding domain is mutated such that the overall alpha-helical structure remains intact to prevent interference with the folding of the histidine kinase domain.

Preferably, the mutant DegS protein differs from the corresponding wild type sequence by one or more mutations selected from, in decreasing order of preference, L99F, L99C, L99D, L99E, L99G, L99H, L99K, L99N, L99P, L99Q, L99R, L99S, L99W or L99Y.

For the purposes of the present invention, the aforementioned numbering is with reference to the wild type DegS protein sequence of Uniprot identifier A0A074LBY4_PAEPO. It is to be noted, as indicated above, that the mutant DegS protein, when disregarding the above mentioned specifically listed mutations, has at least 40%, more preferably at least 46%, more preferably at least 58% and even more preferably 80-100% sequence identity to the amino acid sequence given by Uniprot identifier A0A074LBY4_PAEPO.

The aformenetioned specific mutations fall within the second predicted alpha helix of the DegS Sensor domain. As shown in the examples, such mutations both result in an increase in conjugation competence.

The mutated amino acids for the DegS mutant protein are listed above in increasing order of their respective frequency in natural homologs of DegS proteins. As the invention is interested in providing microorganisms with altered properties of the DegS protein compared to the wild type, the most infrequent alteration is the most preferred one, and preference decreases with increasing frequency of the respective amino acid at the respective position.

The microorganism according to the present invention preferably comprises a mutant degU gene, wherein the degU gene codes for a DegU protein having reduced DNA binding activity and/or lacks a functional DNA binding domain. This is preferably achieved by providing a mutant degU gene coding for a mutant DegU protein, wherein the mutation affects the LuxR-type DNA-binding HTH domain (PF00196). As shown below in the examples, the mere provision of a mutant degU gene already is sufficient to improve conjugation competence.

The DegU protein preferably has a reduced DNA binding activity and/or lacks a functional DNA binding domain. The presence of these traits can be easily identified in the microorganism of the present invention, preferably of genus Paenibacillus, by observing an increase in conjugation competence compared to the corresponding wild type strain.

As described above, the wild type DegU protein comprises a DNA-binding HTH (helix-turn-helix) domain; this domain extends, according to the numbering of the protein sequence with Uniprot identifier E3EBP5, from amino acid position 171 to the end of the sequence. Further information on the DNA binding domain is available from the corresponding Pfam and InterPro databases. For example, for the most preferred wild type DegU protein sequence E3EBP5 the DNA-binding domain is predicted to comprise 4 alpha-helix domains, spanning the positions 180-191, 195-202, 206-221 and 225-235. It is preferred if the DegU protein DNA-binding domain is mutated in the third or fourth, most preferably in the third alpha helical domain. Here, mutations in the protein sequence will generally not influence correct folding and functioning of the remainder of the DegU protein.

Preferably, the DegU protein mutation comprises or consists of, in decreasing order of preference for each alternative a) and b), one or more of:
a) Q218*, Q218K, Q218N, Q218D, Q218R
b) D223*, D223*+M220N, D223*+M220N+E221G, D223*+M220N+V222G, D223*+M220N+E221G+V222G, D223*+M220D, D223*+M220E, D223*+M220H, D223*+M220F, D223*+M220W, D223*+M220S, D223*+M220A,

For the purposes of the present invention, the aforementioned numbering is with reference to the wild type DegU protein sequence of Uniprot identifier E3EBP5. It is to be noted, as indicated above, that the mutant DegU protein, when disregarding the above mentioned specifically listed mutations, has at least 45%, more preferably at least 51%, more preferably at least 54% and even more preferably 73-100% sequence identity to the amino acid sequence given by Uniprot identifier E3EBP5_PAEPS.

Both mutations a) and b) simultaneously fall within the third predicted alpha helix of the DNA binding domain. As shown in the examples, mutations of both type a) and b) result in an increase of conjugation competence.

The mutated amino acids according to alternative a) and b), respectively, are listed above in increasing order of their respective frequency in natural homologs of DegU proteins. As the invention is interested in providing microorganisms with altered properties of the DegU protein compared to the wild type, the most infrequent alteration is the most preferred one, and preference decreases with increasing frequency of the respective amino acid at the respective position.

With the exception of mutation Q218* the aforementioned DegU protein mutations can also be combined. Thus, the invention also pertains to microorganisms comprising a mutant degU gene coding for a mutant DegU protein, wherein the mutation comprises or consists of any of Q218K+D223*, Q218K+M220N+D223*, Q218K+M220N+E221G+D223*, Q218K+M220N+V222G+D223*, Q218K+M220N+E221G+V222G+D223*, Q218K+M220D+D223*, Q218K+M220E+D223*, Q218K+M220H+D223*, Q218K+M220F+D223*, Q218K+M220W+D223*, Q218K+M220S+D223*, Q218K+M220A+D223*, Q218N+D223*, Q218N+M220N+D223*, Q218N+M220N+E221G+D223*, Q218N+M220N+V222G+D223*, Q218N+M220N+E221G+V222G+D223*, Q218N+M220D+D223*, Q218N+M220E+D223*, Q218N+M220H+D223*, Q218N+M220F+D223*, Q218N+M220W+D223*, Q218N+M220S+D223*, Q218N+M220A+D223*, Q218D+D223*, Q218D+M220N+D223*, Q218D+M220N+E221G+D223*, Q218D+M220N+V222G+D223*, Q218D+M220N+E221G+V222G+D223*, Q218D+M220D+D223*, Q218D+M220E+D223*, Q218D+M220H+D223*, Q218D+M220F+D223*, Q218D+M220W+D223*, Q218D+M220S+D223*, Q218D+M220A+D223*, Q218R+D223*, Q218R+M220N+D223*, Q218R+M220N+E221G+D223*, Q218R+M220N+V222G+D223*, Q218R+M220N+E221G+V222G+D223*, Q218R+M220D+D223*, Q218R+M220E+D223*, Q218R+M220H+D223*, Q218R+M220F+D223*, Q218R+M220W+D223*, Q218R+M220S+D223*, Q218R+M220A+D223*, in the numbering of the sequence according to the Uniprot identifier E3EBP5. The microorganism according to the present invention preferably comprises a mutant Spo0A gene, wherein the mutation is located in the DNA binding or receiver domain and results in a reduction or elimination of phosphorylation of the Spo0A protein. As shown below in the examples, the mere provisioning of a mutant Spo0A gene is already sufficient to improve conjugation competence.

The mutant Spo0A protein preferably lacks a functional DNA binding or receiver domain. The presence of these traits can be easily identified in the microorganism of the present invention, preferably of genus Paenibacillus, by observing an increase in conjugation competence compared to the corresponding wild type strain, and can be easily achieved, for example by introducing a mutation in the Spo0A C-terminal domain (IPR014879).

As described above, the wild type Spo0A protein comprises a Sporulation initiation factor Spo0A C terminal domain; this domain extends, according to the numbering of the protein sequence with Uniprot identifier A0A074LZY6_PAEPO, from amino acid position 158 to 261. Further information on the Spo0A C-terminal domain is available from the aforementioned corresponding Pfam and InterPro databases.

Preferably, the mutation of the mutant Spo0A protein consists of or comprises any of
A257V, more preferably A257S,
I161R, more preferably I161L,
in decreasing order of preference: A257S+I161I, A257A+I161L, A257V+I161I, A257S+I161F or A257A+I161R.

For the purposes of the present invention, the aforementioned numbering is with reference to the wild type Spo0A protein sequence of Uniprot identifier A0A074LZY6_PAEPO. It is to be noted, as indicated above, that the mutant Spo0A protein, when disregarding the above mentioned specifically listed mutations, has at least 55%, more preferably at least 60%, more preferably at least 62%, more preferably at least 70%, even more preferably 80-100% and even more preferably 95-100% sequence identity to the amino acid sequence given by Uniprot identifier A0A074LZY6_PAEPO.

Preferably, the mutant Spo0A protein comprises one of the two aforementioned mutations at position 257, i.e. A257V or, more preferably, A257S. This position falls within the last predicted alpha helix of the Spo0A C-terminal domain. Also preferably the mutant Spo0A protein comprises one of the two aforementioned mutations at position 161, i.e. I161R or, more preferably I161L. This position falls within the first predicted alpha helix of the Spo0A C-terminal domain. Further preferably the mutant Spo0A protein comprises any of the aforementioned respective mutations at each of the aforementioned positions, i.e. in decreasing order of preference: A257S+I161I, A257A+I161L, A257V+I161I, A257S+I161F or A257A+I161R. The mutated amino acids of the double mutants are listed in increasing order of their respective frequency in natural homologs of Spo0A proteins. As the invention is interested in providing microorganisms with altered properties of the Spo0A protein compared to the wild type, the most infrequent alteration is the most preferred one, and preference decreases with increasing frequency of the respective amino acid at the respective position.

The invention preferably provides a microorganism, wherein
the expression of the wild type (a) degU and degS genes or, respectively (b) spo0A gene is less than or the same as the expression of the mutant (a) degU and degS genes or, respectively (b) spo0A gene, or
the expression of the wild type (a) degU and degS genes or, respectively (b) spo0A gene is inhibited or eliminated during expression of the mutant (a) degU and degS genes or, respectively (b) spo0A gene.

Such relative overexpresion of the mutant degS, degU and spo0A genes, respectively, over their corresponding wild type equivalent can be achieved in a first and preferred alternative by a microorganism in which the respective genes coding for wild type DegU, DegS and Spo0A proteins, respectively, have been inactivated and gene(s) coding for the respective mutant proteins have been introduced. In such an alternative, the microorganism preferably comprises either
a) a mutant degU and a mutant degS gene according to the invention, and the wild type degU and degS genes are functionally inactivated, removed or is replaced by the mutant genes, or
b) a mutant spo0A gene according to the invention, and the wild type spo0A gene is functionally inactivated, removed or is replaced by the mutant gene.

In a second alternative, the respective wild type gene(s) is/are still present in the microorganism of the present invention. Such microorganisms are particularly beneficial by allowing to switch between the wild type and the mutant behaviour when the wild type or mutant gene or genes is/are brought under the control of a regulatable promoter. This way competence can be increased selectively during a process of intended nucleic acid transfer, whereas wild type low conjugation competence can be maintained in all other stages, thereby for example beneficially limiting horizontal gene transfer during a fermentation process. Correspondingly, a microorganism according to the present invention is provided wherein
the mutant degU and degS gene or the mutant spo0A gene, respectively, is operably linked to an inducible or repressible promoter, and/or
the wild type degU and degS gene or the mutant spo0A gene, respectively, is operably linked to a repressible or inducible promoter,
   preferably such that expression of the mutant degU and degS gene or the mutant spo0A gene, respectively, can be increased or decreased at will relative to the expression of the corresponding wild type genes.

Preferably the mutant degU and degS or spo0A genes, respectively, are provided in respective expression cassettes located on an extrachromosomal nucleic acid, and wherein the extrachromosomal nucleic acid further comprises a counterselectable marker. As described below, such extrachromosomal nucleic acid allows to confer increased competence to the microorganism for a selected period of time. In particular, such extrachromosomal nucleic acid advantageously can be removed from the microorganism after a conjugation event, for example before creation of a cell bank sample of the production strain obtained by conjugation. Counterselectable markers are known to the person skilled in the art and are described, for example, in WO2021061694.

The microorganism according to the present invention preferably is selected from the taxonomic rank of
of phylum Firmicutes, class Bacilli, Clostridia or Negativicutes,
more preferably of order Bacillales, Clostridiales, Thermoanaerobacterales, Thermosediminibacterales or Selenomonadales,
more preferably of family Bacillaceae, Paenibacillaceae, Pasteuriaceae, Clostridiaceae, Peptococcaceae, Heliobacteriaceae, Syntrophomonadaceae, Thermoanaerobacteraceae, Tepidanaerobacteraceae or Sporomusaceae,
more preferably of genus Alkalibacillus, Bacillus, Geobacillus, Halobacillus, Lysinibacillus, Piscibacillus, Terribacillus, Brevibacillus, Paenibacillus, Thermobacillus, Pasteuria, Clostridium, Desulfotomaculum, Heliobacterium, Pelospora, Pelotomaculum, Caldanaerobacter, Moorella, Thermoanaerobacter, Tepidanaerobacter, Propionispora or Sporomusa,
more preferably of genus Bacillus, Paenibacillus or Clostridium.

Particularly microorganisms of the families Bacillaceae and Paenibacillaceae are important microorganisms in industrial fermentation processes. Furthermore, among microorganisms of such genera are known spore producers.

In agriculture, bacterial spores were used in plant pest control compositions reducing or preventing phytopathogenic fungal or bacterial diseases. Spore biologicals are also applied to improve plants resistance against biotic and abiotic stress, to accelerate the growth of the plant and to increase the yield during plant, fruit or legume harvest. Spore products were applied to leaves, shoots, fruits, roots or plant propagation material as well as to the substrate where the plants are to grow (Toyota K. Bacillus-related Spore Formers: Attractive Agents for Plant Growth Promotion. Microbes Environ. 2015;30 (3): 205-207. doi:10.1264/jsme2.me3003rh). Bochow, H., et al. “Use of Bacillus subtilis as Biocontrol Agent. IV. Salt-Stress Tolerance Induction by Bacillus subtilis FZB24 Seed Treatment in Tropical Vegetable Field Crops, and Its Mode of Action/Die Verwendung von Bacillus subtilis zur biologischen Bekämpfung. IV. Induktion einer Salzstress-Toleranz durch Applikation von Bacillus subtilis FZB24 bei tropischem Feldgemüse und sein Wirkungsmechanismus.” Zeitschrift für Pflanzenkrankheiten und Pflanzenschutz/Journal of Plant Diseases and Protection, vol. 108, no. 1, 2001, pp. 21-30. JSTOR, www.jstor.org/stable/43215378. Accessed 14 Dec. 2020.) (Hashem, Abeer & Tabassum, B. & Abd_Allah, Elsayed. (2019). Bacillus subtilis: A plant-growth promoting rhizobacterium that also impacts biotic stress. Saudi Journal of Biological Sciences. 26. 10.1016/j.sjbs.2019.05.004.)

Furthermore, bacterial spores were applied in the area of nanobiotechnology and building chemistry such as for self-healing concrete (crack healing), mortar stability and reduced water permeability [J.Y. Wang, H. Soens, W. Verstraete, N. De Belie, Self-healing concrete by use of microencapsulated bacterial spores, Cement and Concrete Research, Volume 56, 2014, 139-152, ISSN 0008-8846, https://doi.org/10.1016/j.cemconres.2013.11.009] [Ricca E, Cutting SM. Emerging Applications of Bacterial Spores in Nanobiotechnology. J Nanobiotechnology. 2003;1 (1):6. Published 2003 Dec. 15. doi:10.1186/1477-3155-1-6].

Additionally, bacterial spores were applied in the area of cleaning products, such as for cleaning of laundry, hard surfaces, sanitation and odor control (Caselli E. Hygiene: microbial strategies to reduce pathogens and drug resistance in clinical settings. Microb Biotechnol. 2017 September;10 (5):1079-1083. doi: 10.1111/1751-7915.12755. Epub 2017 Jul. 5) in the clinical and domestic setting. As an example, spores were used in cosmetic compositions such as skin cleaning products (US20070048244), for dishwashing agents (WO2014/107111), pipe degreasers (DE19850012), malodor control of laundry (WO2017/157778 and EP3430113) or the removal of allergens (US20020182184). Spores can also be embedded into non-biogenic matrices to catalyse subsequent matrix breakdown.

In addition, bacterial spores were applied in the area of human and animal nutrition and health. As an example different bacterial strains were applied to broilers as part of antibiotic replacement strategy (Neveling, D.P., Dicks, L.M. Probiotics: an Antibiotic Replacement Strategy for Healthy Broilers and Productive Rearing. Probiotics & Antimicro. Prot. 13, 1-11 (2021). https://doi.org/10.1007/s12602-020-09640-z). Other examples include aquaculture, pigs and many more (Nayak, S.K. (2021), Multifaceted applications of probiotic Bacillus species in aquaculture with special reference to Bacillus subtilis. Rev. Aquacult., 13: 862-906. https://doi.org/10.1111/raq.12503). Applications of bacterial spored for human health are also widely described (e.g. US20180289752; Lee, NK., Kim, WS. & Paik, HD. Bacillus strains as human probiotics: characterization, safety, microbiome, and probiotic carrier. Food Sci Biotechnol 28, 1297-1305 (2019). https://doi.org/10.1007/s10068-019-00691-9).

Particularly preferred are microorganisms of one of the following species:

Paenibacillus species: P. abekawaensis, P. abyssi, P. aceris, P. aceti, P. aestuarii, P. agarexedens, P. agaridevorans, P. alba, P. albidus, P. albus, P. alginolyticus, P. algorifonticola, P. alkaliterrae, P. alvei, P. amylolyticus, P. anaericanus, P. antarcticus, P. antibioticophila, P. antri, P. apiaries, P. apiarius, P. apis, P. aquistagni, P. arachidis, P. arcticus, P. assamensis, P. aurantiacus, P. azoreducens, P. azotifigens, P. baekrokdamisoli, P. barcinonensis, P. barengoltzii, P. beijingensis, P. borealis, P. bouchesdurhonensis, P. bovis, P. brasilensis, P. brassicae, P. bryophyllum, P. caespitis, P. camelliae, P. camerounensis, P. campinasensis, P. castaneae, P. catalpae, P. cathormii, P. cavernae, P. cellulosilyticus, P. cellulositrophicus, P. chartarius, P. chibensis, P. chinensis, P. chinjuensis, P. chitinolyticus, P. chondroitinus, P. chungangensis, P. cineris, P. cisolokensis, P. contaminans, P. cookii, P. crassostreae, P. cucumis, P. curdlanolyticus, P. daejeonensis, P. dakarensis, P. darangshiensis, P. darwinianus, P. dauci, P. dendritiformis, P. dongdonensis, P. donghaensis, P. doosanensis, P. durus, P. edaphicus, P. ehimensis, P. elgii, P. elymi, P. endophyticus, P. enshidis, P. esterisolvens, P. etheri, P. eucommiae, P. faecis, P. favisporus, P. ferrarius, P. filicis, P. flagellatus, P. fonticola, P. forsythiae, P. frigoriresistens, P. fujiensis, P. fukuinensis, P. gansuensis, P. gelatinilyticus, P. ginsengagri, P. ginsengarvi, P. ginsengihumi, P. ginsengiterrae, P. glacialis, P. glebae, P. glucanolyticus, P. glycanilyticus, P. gorillae, P. graminis, P. granivorans, P. guangzhouensis, P. harenae, P. helianthi, P. hemerocallicola, P. herberti, P. hispanicus, P. hodogayensis, P. hordei, P. horti, P. humicus, P. hunanensis, P. ihbetae, P. ihuae, P. ihumii, P. illinoisensis, P. insulae, P. intestini, P. jamilae, P. jilunlii, P. kobensis, P. koleovorans, P. konkukensis, P. konsidensis, P. koreensis, P. kribbensis, P. kyungheensis, P. lactis, P. lacus, P. larvae, P. lautus, P. lemnae, P. lentimorbus, P. lentus, P. liaoningensis, P. limicola, P. lupini, P. luteus, P. lutimineralis, P. macerans, P. macquariensis, P. marchantiophytorum, P. marinisediminis, P. marinum, P. massiliensis, P. maysiensis, P. medicaginis, P. mendelii, P. mesophilus, P. methanolicus, P. mobilis, P. montanisoli, P. montaniterrae, P. motobuensis, P. mucilaginosus, P. nanensis, P. naphthalenovorans, P. nasutitermitis, P. nebraskensis, P. nematophilus, P. nicotianae, P. nuruki, P. oceanisediminis, P. odorifer, P. oenotherae, P. oralis, P. oryzae, P. oryzisoli, P. ottowii, P. ourofinensis, P. pabuli, P. paeoniae, P. panacihumi, P. panacisoli, P. panaciterrae, P. paridis, P. pasadenensis, P. pectinilyticus, P. peoriae, P. periandrae, P. phocaensis, P. phoenicis, P. phyllosphaerae, P. physcomitrellae, P. pini, P. pinihumi, P. pinisoli, P. pinistramenti, P. pocheonensis, P. polymyxa, P. polysaccharolyticus, P. popilliae, P. populi, P. profundus, P. prosopidis, P. protaetiae, P. provencensis, P. psychroresistens, P. pueri, P. puernese, P. puldeungensis, P. purispatii, P. qingshengii, P. qinlingensis, P. quercus, P. radicis, P. relictisesami, P. residui, P. rhizoplanae, P. rhizoryzae, P. rhizosphaerae, P. rigui, P. ripae, P. rubinfantis, P. ruminocola, P. sabinae, P. sacheonensis, P. salinicaeni, P. sanguinis, P. sediminis, P. segetis, P. selenii, P. selenitireducens, P. senegalensis, P. senegalimassiliensis, P. seodonensis, P. septentrionalis, P. sepulcri, P. shenyangensis, P. shirakamiensis, P. shunpengii, P. siamensis, P. silagei, P. silvae, P. sinopodophylli, P. solanacearum, P. solani, P. soli, P. sonchi group, P. sophorae, P. spiritus, P. sputi, P. stellifer, P. susongensis, P. swuensis, P. taichungensis, P. taihuensis, P. taiwanensis, P. taohuashanense, P. tarimensis, P. telluris, P. tepidiphilus, P. terrae, P. terreus, P. terrigena, P. tezpurensis, P. thailandensis, P. thermoaerophilus, P. thermophilus, P. thiaminolyticus, P. tianmuensis, P. tibetensis, P. timonensis, P. translucens, P. tritici, P. triticisoli, P. tuaregi, P. tumbae, P. tundrae, P. turicensis, P. tylopili, P. typhae, P. tyrfis, P. uliginis, P. urinalis, P. validus, P. velaei, P. vini, P. vortex, P. vorticalis, P. vulneris, P. wenxiniae, P. whitsoniae, P. wooponensis, P. woosongensis, P. wulumuqiensis, P. wynnii, P. xanthanilyticus, P. xanthinilyticus, P. xerothermodurans, P. xinjiangensis, P. xylanexedens, P. xylaniclasticus, P. xylanilyticus, P. xylanisolvens, P. yanchengensis, P. yonginensis, P. yunnanensis, P. zanthoxyli, P. zeae,

preferably P. agarexedens, P. agaridevorans, P. alginolyticus, P. alkaliterrae, P. alvei, P. amylolyticus, P. anaericanus, P. antarcticus, P. assamensis, P. azoreducens, P. barcinonensis, P. borealis, P. brassicae, P. campinasensis, P. chinjuensis, P. chitinolyticus, P. chondroitinus, P. cineris, P. curdlanolyticus, P. daejeonensis, P. dendritiformis, P. ehimensis, P. elgii, P. favisporus, P. glucanolyticus, P. glycanilyticus, P. graminis, P. granivorans, P. hodogayensis, P. illinoisensis, P. jamilae, P. kobensis, P. koleovorans, P. koreensis, P. kribbensis, P. lactis, P. larvae, P. lautus, P. lentimorbus, P. macerans, P. macquariensis, P. massiliensis, P. mendelii, P. motobuensis, P. naphthalenovorans, P. nematophilus, P. odorifer, P. pabuli, P. peoriae, P. phoenicis, P. phyllosphaerae, P. polymyxa, P. popilliae, P. rhizosphaerae, P. sanguinis, P. stellifer, P. taichungensis, P. terrae, P. thiaminolyticus, P. timonensis, P. tylopili, P. turicensis, P. validus, P. vortex, P. vulneris, P. wynnii, P. xylanilyticus,
particularly preferred Paenibacillus koreensis, Paenibacillus rhizosphaerae, Paenibacillus polymyxa, Paenibacillus amylolyticus, Paenibacillus terrae, Paenibacillus polymyxa polymyxa, Paenibacillus polymyxa plantarum, Paenibacillus nov. spec epiphyticus, Paenibacillus terrae, Paenibacillus macerans, Paenibacillus alvei,
more preferred Paenibacillus polymyxa, Paenibacillus polymyxa polymyxa, Paenibacillus polymyxa plantarum, Paenibacillus nov. spec epiphyticus, Paenibacillus terrae, Paenibacillus macerans, Paenibacillus alvei,
even more preferred Paenibacillus polymyxa, Paenibacillus polymyxa polymyxa, Paenibacillus polymyxa plantarum and Paenibacillus terrae.

Bacillus species: B. abyssalis, B. acanthi, B. acidiceler, B. acidicola, B. acidiproducens, B. aciditolerans, B. acidopullulyticus, B. acidovorans, B. aeolius, B. aequororis, B. aeris, B. aerius, B. aerolacticus, B. aestuarii, B. aidingensis, B. akibai, B. alcaliinulinus, B. alcalophilus, B. algicola, B. alkalicola, B. alkalilacus, B. alkalinitrilicus, B. alkalisediminis, B. alkalitelluris, B. alkalitolerans, B. alkalogaya, B. altitudinis, B. alveayuensis, B. amiliensis, B. andreesenii, B. andreraoultii, B. aporrhoeus, B. aquimaris, B. arbutinivorans, B. aryabhattai, B. asahii, B. aurantiacus, B. australimaris, B. azotoformans, B. bacterium, B. badius, B. baekryungensis, B. bataviensis, B. benzoevorans, B. beringensis, B. berkeleyi, B. beveridgei, B. bingmayongensis, B. bogoriensis, B. borbori, B. boroniphilus, B. butanolivorans, B. cabrialesii, B. caccae, B. camelliae, B. campisalis, B. canaveralius, B. capparidis, B. carboniphilus, B. casamancensis, B. caseinilyticus, B. catenulatus, B. cavernae, B. cecembensis, B. cellulosilyticus, B. chagannorensis, B. chandigarhensis, B. cheonanensis, B. chungangensis, B. ciccensis, B. cihuensis, B. circulans, B. clausii, B. coagulans, B. coahuilensis, B. cohnii, B. composti, B. coniferum, B. coreaensis, B. crassostreae, B. crescens, B. cucumis, B. dakarensis, B. daliensis, B. danangensis, B. daqingensis, B. decisifrondis, B. decolorationis, B. depressus, B. deramificans, B. deserti, B. dielmoensis, B. djibelorensis, B. drentensis, B. ectoiniformans, B. eiseniae, B. enclensis, B. endolithicus, B. endophyticus, B. endoradicis, B. endozanthoxylicus, B. farraginis, B. fastidiosus, B. fengqiuensis, B. fermenti, B. ferrariarum, B. filamentosus, B. firmis, B. firmus, B. flavocaldarius, B. flexus, B. foraminis, B. fordii, B. formosensis, B. fortis, B. freudenreichii, B. fucosivorans, B. fumarioli, B. funiculus, B. galactosidilyticus, B. galliciensis, B. gibsonii, B. ginsenggisoli, B. ginsengihumi, B. ginsengisoli, B. glennii, B. glycinifermentans, B. gobiensis, B. gossypii, B. gottheilii, B. graminis, B. granadensis, B. hackensackii, B. haikouensis, B. halmapalus, B. halodurans, B. halosaccharovorans, B. haynesii, B. hemicellulosilyticus, B. hemicentroti, B. herbersteinensis, B. hisashii, B. horikoshii, B. horneckiae, B. horti, B. huizhouensis, B. humi, B. hunanensis, B. hwajinpoensis, B. idriensis, B. indicus, B. infantis, B. infernus, B. intermedius, B. intestinalis, B. iocasae, B. isabeliae, B. israeli, B. jeddahensis, B. jeotgali, B. kexueae, B. kiskunsagensis, B. kochii, B. kokeshiiformis, B. koreensis, B. korlensis, B. kribbensis, B. krulwichiae, B. kwashiorkori, B. kyonggiensis, B. lacisalsi, B. lacus, B. lehensis, B. lentus, B. ligniniphilus, B. lindianensis, B. litoralis, B. loiseleuriae, B. lonarensis, B. longiquaesitum, B. longisporus, B. luciferensis, B. luteolus, B. luteus, B. lycopersici, B. magaterium, B. malikii, B. mangrovensis, B. mangrovi, B. mannanilyticus, B. manusensis, B. marasmi, B. marcorestinctum, B. marinisedimentorum, B. marisflavi, B. maritimus, B. marmarensis, B. massiliglaciei, B. massilioanorexius, B. massiliogabonensis, B. massiliogorillae, B. massilionigeriensis, B. massiliosenegalensis, B. mediterraneensis, B. megaterium, B. mesonae, B. mesophilum, B. mesophilus, B. methanolicus, B. miscanthi, B. muralis, B. murimartini, B. nakamurai, B. nanhaiisediminis, B. natronophilus, B. ndiopicus, B. nealsonii, B. nematocida, B. niabensis, B. niacini, B. niameyensis, B. nitritophilus, B. notoginsengisoli, B. novalis, B. obstructivus, B. oceani, B. oceanisediminis, B. ohbensis, B. okhensis, B. okuhidensis, B. oleivorans, B. oleronius, B. olivae, B. onubensis, B. oryzae, B. oryzaecorticis, B. oryzisoli, B. oryziterrae, B. oshimensis, B. pakistanensis, B. panacisoli, B. panaciterrae, B. paraflexus, B. patagoniensis, B. persicus, B. pervagus, B. phocaeensis, B. pichinotyi, B. piscicola, B. piscis, B. plakortidis, B. pocheonensis, B. polygoni, B. polymachus, B. populi, B. praedii, B. pseudalcaliphilus, B. pseudofirmus, B. pseudoflexus, B. pseudomegaterium, B. psychrosaccharolyticus, B. pumilus, B. purgationiresistens, B. qingshengii, B. racemilacticus, B. rhizosphaerae, B. rigiliprofundi, B. rubiinfantis, B. ruris, B. safensis, B. saganii, B. salacetis, B. salarius, B. salidurans, B. salis, B. salitolerans, B. salmalaya, B. salsus, B. sediminis, B. selenatarsenatis, B. senegalensis, B. seohaeanensis, B. shacheensis, B. shackletonii, B. shandongensis, B. shivajii, B. similis, B. simplex, B. sinesaloumensis, B. siralis, B. smithii, B. solani, B. soli, B. solimangrovi, B. solisilvae, B. songklensis, B. spongiae, B. sporothermodurans, B. stamsii, B. subterraneus, B. swezeyi, B. taeanensis, B. taiwanensis, B. tamaricis, B. taxi, B. terrae, B. testis, B. thaonhiensis, B. thermoalkalophilus, B. thermoamyloliquefaciens, B. thermoamylovorans, B. thermocopriae, B. thermolactis, B. thermophilus, B. thermoproteolyticus, B. thermoterrestris, B. thermozeamaize, B. thioparans, B. tianmuensis, B. tianshenii, B. timonensis, B. tipchiralis, B. trypoxylicola, B. tuaregi, B. urumqiensis, B. vietnamensis, B. vini, B. vireti, B. viscosus, B. vitellinus, B. wakoensis, B. weihaiensis, B. wudalianchiensis, B. wuyishanensis, B. xiamenensis, B. xiaoxiensis, B. zanthoxyli, B. zeae, B. zhangzhouensis, B. zhanjiangensis,

preferably Bacillus licheniformis, B. megaterium, B. subtilis, B. pumilus, B. firmus, B. thuringiensis, B. velezensis, B. linens, B. atrophaeus, B. amyloliquefaciens, B. aryabhattai, B. cereus, B. aquatilis, B. circulans, B. clausii, B. sphaericus, B. thiaminolyticus, B. mojavensis, B. vallismortis, B. coagulans, B. sonorensis, B. halodurans, B. pocheonensis, B. gibsonii, B. acidiceler, B. flexus, B. hunanensis, B. pseudomycoides, B. simplex, B. safensis, B. mycoides,
particularly preferred B. amyloliquefaciens, B. licheniformis, B. thuringiensis, B. velezensis, B. subtilis and B. megaterium,
even more preferably B. amyloliquefaciens, B. thuringiensis, B. velezensis and B. megaterium.

Clostridium species: C. autoethanogenum, C. beijerinckii, C. butyricum, C. carboxidivorans, C. disporicum, C. drakei, C. ljungdahlii, C. kluyveri, C. pasteurianum, C. propionicum, C. saccharobutylicum, C. saccharoperbutylacetonicum, C. scatologenes, C. tyrobutyricum, preferably C. butyricum, C. pasteurianum and/or C. tyrobutyricum, C. aerotolerans, C. aminophilum, C. aminvalericum, C. celerecrescens, C. asparagforme, C. bolteae, C. clostridioforme, C. glycyrrhizinilyticum, C. (Hungatela) hathewayi, C. histolyticum, C. indolis, C. leptum, C. (Tyzzerella) nexile, C. perfringens, C. (Erysipelatoclostridium) ramosum, C. scindens, C. symbiosum, Clostridium saccharogumia, Clostridium sordelli, Clostridium clostridioforme, C. methylpentosum, C. islandicum and all members of the Clostridia clusters IV, XIVa, and XVIII, particularly preferred C. butyricum.

Some suitable Bacillus and Paenibacillus strains are described and deposited in the following international patent applications; spores of such microorganisms or pesticidally active variants of any thereof can be incorporated as spores of the composition according to the invention: WO2020200959: Bacillus subtilis or Bacillus amyloliquefaciens QST713 deposited under NRRL Accession No. B-21661 or a fungicidal mutant thereof. Bacillus subtilis QST713, its mutants, its supernatants, and its lipopeptide metabolites, and methods for their use to control plant pathogens and insects are fully described in U.S. Pat. Nos. 6,060,051, 6,103,228, 6,291,426, 6,417,163 and 6,638,910. In these patents, the strain is referred to as AQ713, which is synonymous with QST713; WO2020102592: Bacillus thuringiensis strains NRRL B-67685, NRRL B-67687, and NRRL B-67688; WO2019135972: Bacillus megatherium having the deposit accession number NRRL B-67533 or NRRL B-67534; WO2019035881: Paenibacillus sp. NRRL B-50972, Paenibacillus sp. NRRL B-67129, Bacillus subtilis strain QST30002 deposited under accession no. NRRL B-50421, and Bacillus subtilis strain NRRL B-50455; WO2018081543: Bacillus psychrosaccharolyticus strain deposited under ATCC accession number PT A-123720 or PT A-124246; WO2017151742: Bacillus subtilis assigned the accession number NRRL B-21661; WO2016106063: Bacillus pumilus NRLL B-30087; WO2013152353: Bacillus sp. deposited as CNMC 1-1582; WO2013016361: Bacillus sp. strain SGI-015-F03 deposited as NRRL B-50760, Bacillus sp. strain SGI-015-H06 deposited as NRRL B-50761; WO2020181053: Paenibacillus sp. NRRL B-67721, Paenibacillus sp. NRRL B-67723, Paenibacillus sp. NRRL B-67724, Paenibacillus sp. NRRL B-50374.

The invention also provides a method of increasing conjugation competence of a microorganism, comprising the step of providing, in the microorganism, either
a) a mutant DegS protein and preferably a mutant DegU protein according to the present invention, or
b) a mutant Spo0A protein according to the present invention.

As described herein, the selective provision of such mutant proteins advantageously improves conjugation competence of the microorganism.

Correspondingly the invention also provides a method of transferring genetic material between two microorganisms, comprising
1) providing, in a first microorganism,
a) a mutant DegS protein according to the present invention and preferably a mutant DegU protein according to the present invention, or
b) a mutant Spo0A protein according to the present invention, and
2) conjugating the first microorganism with a conjugation competent second microorganism, wherein the first microorganism comprises, before step 2, the genetic material to be transferred.

As described herein, the provision of (a) the mutant DegS protein, preferably together with the mutant DegU protein and in any case without the mutant Spo0A protein or (b) the mutant Spo0A protein without the mutant DegS and optionally DegU protein results in an increased conjugation competence of the first microorganism (“target microorganism”). When this target microorganism is brought into contact with a conjugation competent second microorganism carrying the nucleic acid to be transferred (“donor microorganism”), said nucleic acid is conjugatively transferred with high efficiency.

In the transfer method of the present invention, preferably the mutant degU and degS or spo0A genes, respectively, are provided in respective expression cassettes located on an extrachromosomal nucleic acid in the target microorganism, and wherein the extrachromosomal nucleic acid further comprises a counterselectable marker and the transfer method further comprises the step of counterselecting against the counterselectable marker. As described herein, by using a counterselectable marker the extrachromosomal nucleic acid conferring conjugation competence can be removed, thereby increasing stability of the microorganism after uptake of the conjugatively transferred nucleic acid and preventing or limiting further horizontal gene transfer.

Correspondingly the invention also provides an expression vector, comprising an expression cassette for expression of a counterselectable marker, and
a) mutant degS gene according to the invention and preferably a mutant degU gene according to the invention, or
b) a mutant spo0A gene according to the invention.

Such expression vector advantageously aids in providing the respective mutant gene or genes for expression in a target microorganism intended as recipient of a heterologous nucleic acid.

The invention also provides the use of either
a) a mutant degS gene according to the invention and preferably a mutant degU gene according to the invention or
b) a mutant spo0A gene according to the invention,
   for increasing conjugation competence of a microorganism selected from any of the taxonomic ranks of preferred microorganisms listed herein.

By using the respective gene or genes, or by using the corresponding mutant proteins, conjugation competence of a microorganism can be advantageously be improved.

Aspects of the invention is hereinafter further described by way of non-limiting examples.

### EXAMPLES

### Example 1: Mutant Generation

### Strains and Cultivation Conditions

A list of strains used for targeted integration of point mutations by CRISPR Cas9 in P. polymyxa is shown in table 1. Targeted point mutations in wildtype strain P. polymyxa DSM365 were integrated according to the CRISPR Cas9 procedure described in Rütering et. al (Rütering et al., Tailor-made exopolysaccharides-CRISPR-Cas9 mediated genome editing in Paenibacillus polymyxa. Synth Biol (Oxf). 2017 Dec. 21;2(1):ysx007. doi: 10.1093/synbio/ysx007). DSM 365 was obtained from the German Collection of Microorganisms and Cell Culture (DSMZ), Braunschweig, Germany. Plasmid cloning and multiplication were performed in either E. coli DH5α or Turbo from NEB (New England Biolabs, USA). Transformation of P. polymyxa was performed by conjugation mediated by E. coli S17-1 (DSMZ). The strains were grown in LB media (10 g/L tryptone peptone, 5 g/L yeast extract, 5 g/L NaCl). For plate media, 1.5% agar was used. Whenever necessary, the media was supplemented with 50 μg/ml neomycin and/or 20 μg/mL polymyxin for counterselection of positive transformants and to get rid of E. coli after the conjugation procedure. P. polymyxa was grown at 30° C. and 250 rpm while E. coli at 37° C. and 250 rpm, unless stated otherwise. The strains were stored as cryo culture with 24% glycerol and kept at −80 C for longer storage.

TABLE 1
List of strains used for CRISPR Cas9 mediated construction
of targeted point-mutations in P. polymyxa DSM365

| Strain | Description | Reference | Use |
|---|---|---|---|
| E. coli | F− Φ80lacZΔM15 Δ | NEB | High copy plasmid |
| DH5α | (lacZYA-argF) 169 recA1 | | amplification of pCasPP |
| | endA1 hsd R17 (rK−, K+) | | before transformation |
| | phoA supE44 λ− thi-1 | | in S17-1 |
| | gyrA96 relA1 | | |
| E. coli | F′ proA+B+ laclq | NEB | High copy plasmid |
| Turbo | ΔlacZM15/fhuA2 Δ(lac-proAB) | | amplification of pCasPP |
| | glnV galK16 galE15 | | before transformation |
| | R(zgb-210::Tn10)TetS | | in S17-1 |
| | endA1 thi-1 Δ(hsdS-mcrB)5 | | |
| E. coli | recA pro hsdR | ATCC 47055 | Conjugation |
| S17-1 | RP42Tc::Mu-Km::Tn7 | (DSM 9079) | strain: donor |
| | integrated into the | | |
| | chromosome | | |
| P. polymyxa | Wild type (public strain) | DSM365/BASF # | Target strain for |
| DSM365 | | LU21039 | modification |

### Conjugation

Conjugation was performed between P. polymyxa (recipient strain) and E. coli S17-1 harboring the plasmid of interest (donor strain) according to the CRISPR Cas9 procedure described in Rütering et al. 2017 (Rütering M, Cress BF, Schilling M, Rühmann B, Koffas MAG, Sieber V, Schmid J. Tailor-made exopolysaccharides-CRISPR-Cas9 mediated genome editing in Paenibacillus polymyxa. Synth Biol (Oxf). 2017 Dec. 21;2(1):ysx007. doi: 10.1093/synbio/ysx007. PMID: 32995508; PMCID: PMC7445874). Confirmation of the correct conjugants was performed by colony PCR and sequencing of DNA fragments. Plasmid curing was performed by 1:100 subculturing of the positive mutant in LB liquid media at 37° C.

### Plasmid Construction

Targeted point mutations were achieved by CRISPR-Cas9 mediated system. Selected gRNA sequences were chosen based on their closest proximity to the targeted positions within degU, degS, or spo0A genes. The plasmids were assembled by isothermal Gibson Assembly. Desired point mutations were introduced from the primers used for PCR of the homology flanks. For degS and spo0A, several silent mutations were also introduced in the primers to improve efficiency of the system. Homology flanks were obtained by PCR of P. polymyxa genomic DNA, about 1 kbp upstream and downstream of the targeted nucleotides. E. coli DH5α or Turbo was transformed with the Gibson assembly mixture and plated on LB plate containing 50 μg/ml neomycin. Screening of the positive colonies was done by colony PCR. Plasmids were isolated by miniprep and verified by sequencing for further confirmation. The correct plasmid was used to transform E. coli S17-1 which would then mediate the transformation to P. polymyxa.

Using the pCasPP vector system and homologues flanks carrying 1000 bp, each, of the surrounding genomic sequences flanking the targeted point mutation region, the following mutations were generated (table 2):

TABLE 2
List of mutant strains and associated spacer sequences used for genome editing by
CRISPR Cas9. SNP = single nucleotide polymorphism, nt = nucleotide.

| | | Modification | |
|---|---|---|---|
| strain | gene | (Amino acid / nucleotide position) | spacer sequence 5′→3′ |
| DSM365 | degU | SNP: | CAGCAGTATTTTGCAAAAAA |
| | | degU Q218* (nt. pos. 652 C→T) | |
| DSM365 | degU | Insertion: | CAGCAGTATTTTGCAAAAAA |
| | | degU D223* + M220N + E221G + V222G | |
| | | (Insertion at nt. pos. 658 A→AA | |
| DSM365 | degS | SNP: | TGTGATGATTTTCCGCGAGA |
| | | degS L99F (nt. pos. 295 C→T) | |
| DSM365 | spoOA | SNP: | TAAGCTGAGAATTGAGAACA |
| | | spo0A A257V (nt. pos. 770° C.→T) | |
| DSM365 | degS + | Double mutant | See above |
| | degU | degS L99F + degU Q218* | |
| DSM365 | spoOA + | Triple mutant | See above |
| | degS + | spo0A A257V + degS L99F + degU Q218* | |
| | degU | | |

### Example 2: Genetic Competence Evaluation

Genetic competence of the different variants was evaluated by conjugating the cured strains with E. coli S17-1 harboring pCasPP plasmid as the donor strain, following the protocol as described above. The plasmid contains SpCas9 gene expressed under control of constitutive sgsE promoter from Geobacillus stearothermophilus and does not contain any gRNA targeting P. polymyxa genome. To obtain countable colonies, serial dilution was prepared before plating the conjugated strains onto selection LB plate with antibiotics. Colony forming units from each strain were then normalized to its respective OD₆₀₀ used for conjugation. The increase in competency is calculated based on the number of CFUs after conjugation, as compared to the wild type strain.

1. A microorganism comprising either
a) a mutant degS gene and optionally a mutant degU gene, or
b) a mutant spo0A gene,
wherein the microorganism exhibits increased conjugation competence relative to the corresponding wild type strain.

2. The microorganism according to claim 1, comprising a mutant degS gene, wherein
the degS gene codes for a DegS protein lacking a functional single binding domain, a functional phosphoacceptor domain and/or a functional ATPase domain and/or
the degS gene codes for a DegS protein, wherein the mutation comprises or consists of L99F, L99C, L99D, L99E, L99G, L99H, L99K, L99N, L99P, L99Q, L99R, L99S, L99W or L99Y.

3. The microorganism according to claim 1, comprising a mutant degU gene, wherein
the degU gene codes for a DegU protein having reduced DNA binding activity and/or lacks a functional DNA binding domain, and/or
the degU gene codes for a DegU protein, wherein the mutation comprises or consists of one or more of:
a) Q218*, Q218K, Q218N, Q218D, or Q218R, or
b) D223*, D223*+M220N, D223*+M220N+E221G, D223*+M220N+V222G, D223*+M220N+E221G+V222G, D223*+M220D, D223*+M220E, D223*+M220H, D223*+M220F, D223*+M220W, D223*+M220S, or D223*+M220A.

4. The microorganism according to claim 1, comprising a mutant spo0A gene, wherein
a) the mutation is located in the DNA binding or receiver domain and results in a reduction or elimination of phosphorylation of the Spo0A protein and/or a reduction or elimination of dimerisation, and/or
b) the mutation consists of or comprises any of
A257V,
I161R,
A257S+I161I, A257A+I161L, A257V+I161I, A257S+I161F or A257A+I161R.

5. The microorganism according to claim 1, wherein
the expression of the wild type (a) degU and degS genes or, respectively (b) spo0A gene is less than the expression of the mutant (a) degU and degS genes or, respectively (b) spo0A gene, or
the expression of the wild type (a) degU and degS genes or, respectively (b) spo0A gene is inhibited or eliminated during expression of the mutant (a) degU and degS genes or, respectively (b) spo0A gene.

6. The microorganism according to claim 5, wherein the microorganism comprises either
a) a mutant degU and a mutant degS gene, and the wild type degU and degS genes are functionally inactivated, removed or is replaced by the mutant genes, or
b) a mutant spo0A gene, and the wild type spo0A gene is functionally inactivated, removed or is replaced by the mutant gene.

7. The microorganism according to claim 1, wherein the mutant degU and degS or spo0A genes, respectively, are provided in respective expression cassettes located on an extrachromosomal nucleic acid, and wherein the extrachromosomal nucleic acid further comprises a counterselectable marker.

8. The microorganism according to claim 1, wherein the microorganism is selected from a taxonomic rank selected from the group consisting of
phylum Firmicutes, class Bacilli, Clostridia or Negativicutes,
order Bacillales, Clostridiales, Thermoanaerobacterales, Thermosediminibacterales or Selenomonadales,
family Bacillaceae, Paenibacillaceae, Pasteuriaceae, Clostridiaceae, Peptococcaceae, Heliobacteriaceae, Syntrophomonadaceae, Thermoanaerobacteraceae, Tepidanaerobacteraceae or Sporomusaceae,
genus Alkalibacillus, Bacillus, Geobacillus, Halobacillus, Lysinibacillus, Piscibacillus, Terribacillus, Brevibacillus, Paenibacillus, Thermobacillus, Pasteuria, Clostridium, Desulfotomaculum, Heliobacterium, Pelospora, Pelotomaculum, Caldanaerobacter, Moorella, Thermoanaerobacter, Tepidanaerobacter, Propionispora or Sporomusa, and
genus Bacillus, Paenibacillus or Clostridium.

9. A method of increasing conjugation competence of a microorganism, comprising the step of providing, in the microorganism, the mutant DegS protein according to claim 2 and optionally a mutant DegU protein.

10. A method of transferring genetic material between two microorganisms, comprising
1) providing, in a first microorganism,
a mutant DegS protein according to claim 2 and optionally a mutant DegU protein, and
2) conjugating the first microorganism with a conjugation competent second microorganism,
wherein the first microorganism comprises, before step 2, the genetic material to be transferred.

11. The method according to claim 10, wherein the method further comprises the step of counterselecting against the counterselectable marker.

12. An expression vector, comprising an expression cassette for expression of a counterselectable marker, and the mutant degS gene according to claim 2 and, optionally, a mutant degU gene.

13. A method of using the mutant degS gene according to claim 2 and optionally a mutant degU gene, the method comprising using the mutant degS gene and optionally the mutant degU gene for increasing conjugation competence of a microorganism selected from a taxonomic rank selected from the group consisting of
phylum Firmicutes, class Bacilli, Clostridia or Negativicutes,
order Bacillales, Clostridiales, Thermoanaerobacterales, Thermosediminibacterales or Selenomonadales,
family Bacillaceae, Paenibacillaceae, Pasteuriaceae, Clostridiaceae, Peptococcaceae, Heliobacteriaceae, Syntrophomonadaceae, Thermoanaerobacteraceae, Tepidanaerobacteraceae or Sporomusaceae,
genus Alkalibacillus, Bacillus, Geobacillus, Halobacillus, Lysinibacillus, Piscibacillus, Terribacillus, Brevibacillus, Paenibacillus, Thermobacillus, Pasteuria, Clostridium, Desulfotomaculum, Heliobacterium, Pelospora, Pelotomaculum, Caldanaerobacter, Moorella, Thermoanaerobacter, Tepidanaerobacter, Propionispora or Sporomusa, and
genus Bacillus, Paenibacillus or Clostridium.

14. The method of claim 13, wherein:
the mutant degU gene codes for a DegU protein having reduced DNA binding activity and/or lacks a functional DNA binding domain, and/or
the mutant degU gene codes for a DegU protein, wherein the mutation comprises or consists of-one or more of:
a) Q218*, Q218K, Q218N, Q218D, or Q218R, or
b) D223*, D223*+M220N, D223*+M220N+E221G, D223*+M220N+V222G, D223*+M220N+E221G+V222G, D223*+M220D, D223*+M220E, D223*+M220H, D223*+M220F, D223*+M220W, D223*+M220S, or D223*+M220A.

15. A method of using the mutant Spo0A protein according to claim 4, the method comprising using the mutant degS gene and optionally the mutant degU gene for increasing conjugation competence of a microorganism selected from a taxonomic rank selected from the group consisting of
phylum Firmicutes, class Bacilli, Clostridia or Negativicutes,
order Bacillales, Clostridiales, Thermoanaerobacterales, Thermosediminibacterales or Selenomonadales,
family Bacillaceae, Paenibacillaceae, Pasteuriaceae, Clostridiaceae, Peptococcaceae, Heliobacteriaceae, Syntrophomonadaceae, Thermoanaerobacteraceae, Tepidanaerobacteraceae or Sporomusaceae,
genus Alkalibacillus, Bacillus, Geobacillus, Halobacillus, Lysinibacillus, Piscibacillus, Terribacillus, Brevibacillus, Paenibacillus, Thermobacillus, Pasteuria, Clostridium, Desulfotomaculum, Heliobacterium, Pelospora, Pelotomaculum, Caldanaerobacter, Moorella, Thermoanaerobacter, Tepidanaerobacter, Propionispora or Sporomusa, and
genus Bacillus, Paenibacillus or Clostridium.

16. A method of increasing conjugation competence of a microorganism, comprising the step of providing, in the microorganism, the mutant Spo0A protein according to claim 4.

17. A method of transferring genetic material between two microorganisms, comprising
1. providing, in a first microorganism,
the mutant Spo0A protein according to claim 4, and
2) conjugating the first microorganism with a conjugation competent second microorganism,
wherein the first microorganism comprises, before step 2, the genetic material to be transferred.

18. The method according to claim 17, wherein the method further comprises the step of counterselecting against the counterselectable marker.

19. An expression vector, comprising an expression cassette for expression of a counterselectable marker, and the mutant Spo0A protein according to claim 4.

20. The microorganism according to claim 1, comprising a mutant spo0A gene, wherein the mutation consists of or comprises A257S or I161L.
